(19) United States
(12) Patent Application Publication
(10) Pub. No.: US 2023/0189637 A1
(43) Pub. Date: Jun. 15, 2023

(54) ELECTRONICALLY ACTIVE, SOLVENT RESISTANT ORGANIC FILMS PROCESSED FROM ALCOHOL OR AQUEOUS MEDIA

(71) Applicant: UTI LIMITED PARTNERSHIP, Calgary (CA)

(72) Inventors: Cayley HARDING, Calgary (CA); Gregory C. WELCH, Calgary (CA)

(21) Appl. No.: 17/922,226

(22) PCT Filed: Apr. 30, 2021
(86) PCT No.: PCT/CA2021/050603

§ 371 Date: Oct. 28, 2022

Related U.S. Application Data
(60) Provisional application No. 63/019,012, filed on May 1, 2020.

(51) Int. Cl. H10K 85/60; G01N 31/22; G01N 21/78; H10K 30/50; H10K 71/15; H10K 30/85

(52) U.S. Cl. CPC H10K 85/621; G01N 21/78; G01N 31/227; H10K 30/50; H10K 30/85; H10K 71/15; H10K 71/13

(57) ABSTRACT

Thin films of organic semiconducting material comprising perylene diimide small molecules with pyrrolic N—H bonds. Films are prepared using green solvents including water and alcohols. The films can be solvent-resistant and generally range in thickness from 10 to 1000 nm. Perylene diimide molecules are dissolved in solvent by addition of a base to polarize the pyrrolic N—H bond believed to generate an ionic salt in alcohol or aqueous solution. Devices containing such films are provided. Methods of making films and methods of using films in OPV device applications and in amine sensors are provided.

### FIELD OF THE INVENTION

The invention relates to aqueous or alcohol formulations that can be printed or coated to form organic films that are electronically active and solvent resistant. The films are relevant to the field of organic electronics with potential use in solar cells, light emitting diodes, sensors, transistors, electrochromics, thermoelectrics, photocatalysis (photoanodes and photocathodes).

### BACKGROUND

Perylene diimide (PDI) dyes are stable colorants with diverse uses as textile dyes, print media, and automotive paint. [1, 2] The extended π-conjugated structure yields strong visible light absorption and deep lying frontier molecular orbital (FMO) energy levels. [3-5] PDI molecules self-assemble into structures with strongly overlapping π-stacks making the chromophore useful as an electron transport material in optoelectronic devices. [6-8] The PDI building block has been used to prepare functional materials for use in electronic devices including photovoltaics [9], light emitting diodes [10], and field-effect transistors [11]. The self-assembly properties of PDI molecules can be further optimized by modifying the core at the bay, headland, and/or imide positions. Such modification allows tuning of its morphology in the solid-state for realizing high organic electronic device performance. [12, 13]

Both the molecular properties and self-assembly in thin films can be controlled via heteroatom annulation at the bay position of the PDI chromophore. Sulphur [14, 15], selenium [16, 17], and nitrogen [18, 19] annulation have all been explored to create new classes of PDIs. [20, 21] U.S. Pat. 6,491,749 (Langhals et al.), for example, relates to core-extended perylene diimides, including certain N-annulated PDIs.

N-annulation destabilizes the FMO energy levels of PDIs [22-25] and provides an extra site for side-chain engineering. [26, 27] These alterations have led to development of PDIs as non-fullerene acceptors (NFAs) for organic photovoltaic (OPV) applications. In particular, OPV devices based on these NFAs exhibit large open circuit voltages > 1 V and can be roll-to-roll coated using non-halogenated solvents. [28-32] OPV devices often rely on processing functional materials into thin films, for example, by spin-coating or slot-die coating methods.

Concern about adverse health and environmental effects of the materials, processing solvents, and devices at end of use is mounting. [33-36] For example, halogenated aromatic solvents such as 1,2-dichlorobenzene and chlorobenzene are often used for processing such materials despite their toxicity and low vapor pressure. Such solvents pose safety hazards to human and environment health and as such, large scale industrial production can no longer target methods that require these halogenated and aromatic solvents.

Stringent requirements on the solubility, uniformity of film formation, and self-assembly into ideal nano-morphologies [37] have made aromatic and halogenated solvents the most common processing solvents for device fabrication with standard alkylated π-conjugated materials. [38, 39] The molecule and solvent can no longer be thought of independently optimizable parameters, making device fabrication with green solvents a tremendous challenge. [40, 41]

There is a significant need in the art to develop organic semiconductor materials that can be processed from green solvents. [42] There is also a significant need in the art for functional films that are solvent resistant to enable the manufacture of multi-layer devices. [43] Film solvent resistance is a particularly difficult challenge in view of the more limited solvent palette that is typically employed in so-called “orthogonal processing” (i.e. forming multilayer organic films using sequentially immiscible solvents). A method that has been reported to provide solvent resistance for orthogonal processing is to modify a semiconducting polymer with a cleavable or crosslinkable side-chain. [43-45] Solvent resistance imparted by cleavable side-chains is, however, inherently not atom economical. Solvent resistance imparted by crosslinkable side-chains requires post-deposition treatment, such as thermal or UV annealing.

Recently, the use of a cleavable ester [40] or acid [41] modified alkyl chain appended to the conjugated backbone of certain materials has been reported to provide water-processable materials that form solvent-resistant film. In one case, a conjugated thiophene-based polymer is derivatized with a cleavable side chain containing ester groups. [40] Upon treatment with KOH/methanol, a water-soluble dicarboxylic acid salt of the conjugated polymer is reported to be formed which can be processed using water to form a film. UV-treatment of the film results in side-chain cleavage rendering the film solvent resistant. In the second case, a water-soluble conjugated thiophene-based polymer derivatized with side-chains carrying carboxylate salts is processed into a film [41]. Mild acid treatment to convert the salts into the corresponding carboxylic acids is reported to render the film impervious to various solvents. The later process, has features of the process of acid dyeing, where a dye molecule containing acidic functional groups (typical sulfonic acid groups) is deprotonated to increase aqueous solubility of the dye. A textile is then immersed in the solution of the anionic dye and exposed to acid to protonate the dye and permanently color the textile. Acid dyeing is a robust method to generate highly resistant colored fabrics while still retaining high solubility in water. [46]

The present invention provides materials and methods using N-annulated PDI materials to make water- or alcohol-processable materials from which solvent-resistant films can be formed. In this method, N-annulated PDI materials are rendered alcohol or water soluble by deprotonation of the pyrrolic N-H site. In contrast to the methods noted above, on drying the film spontaneously protonates to form a solvent resistant semiconducting film.

### SUMMARY OF THE INVENTION

The invention relates to organic semiconducting film comprising perylene diimide small molecules with pyrrolic N-H bonds. In embodiments, the films are solvent-resistant. In embodiments, the films range in thickness from 10 to 1000 nm. In embodiments, the films are processed from alcohol or aqueous formulations in which the perylene diimide molecules are dissolved. It is believed that alcohol or aqueous solubility is enabled through addition of a base to polarize the pyrrolic N-H bond giving an ionic salt in alcohol or aqueous solution. In embodiments, the alcohol or aqueous formulations herein are inks suitable for printing, for example with an ink-jet printer or slot-die coater.

In embodiments, the invention relates to the formation of thin films, for example, useful as organic semiconducting elements in OPV devices. In embodiments, the invention relates to films, formulations and ink for making films and to methods of making films. In embodiments, the invention relates to organic semiconducting elements in OPV devices which are thin films comprising perylene diimide small molecules with pyrrolic N—H bonds prepared from alcohol or aqueous formulations as described herein.

Films are prepared from N-annulated perylene diimides which contain at least one pyrrole N—H bond. Films are formed from aqueous or alcoholic film-precursor solutions in which the N-annulated PDI compound is dissolved in the water and/or alcohol solvent by addition of base. At least sufficient base is added to the solvent containing the N-annulated PDI compound to dissolve the N-annulated PDI compound in the solvent. It is believed that addition of base, deprotonates the pyrrole N—H group of the NPDI compound to form a soluble NPDI anion.

More specifically, the invention relates to a method for making a thin film of N-annulated perylene diimides, for example, useful in OPV devices. The method comprises dissolving a selected N-annulated PDI compound having at least one pyrrole N—H bond in a solvent selected from water, a C₁-C₆ alcohol or a miscible mixture thereof. A selected amount of the N-annulated PDI compound is dissolved in the solvent by adding base to the solvent until the selected amount of the N-PDI compound in the solvent dissolves. In embodiments, a film is formed from the solution containing the dissolved N-annulated PDI compound. It is believed that the N-annulated PDI compound is deprotonated on dissolution in the solvent. The solution formed is thus a film-precursor solution. In some embodiments, the solution formed is an ink which can be used in appropriate printing devices. After the solution is deposited or printed on a selected substrate, solvent is removed, typically at room temperature at ambient pressure. Dependent upon the method used to form a film, the N-PDI and the solvents used, the solution deposited may be heated or subjected to sub-atmospheric pressure to enhance drying (i.e., solvent removal). Vacuum and heat applied for removal of solvent should not deform the film or be detrimental to the substrate. On removal of solvent, the N-annulated PDI is believed to return to its protonated form.

A film may contain one or more N-annulated PDI compounds each having a pyrrole N—H group.

In embodiments, the N-annulated PDI compound is a compound of Formula I:

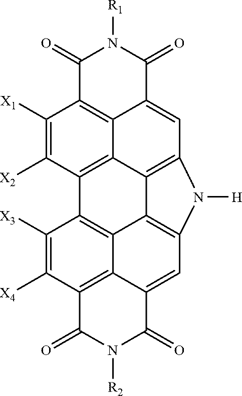

wherein,
R₁ and R₂ are independently a substituted or unsubstituted C₁ to C₁₈ linear or branched alkyl; and
X₁-X₄ are independently selected from H, a C₁-C₆ substituted or unsubstituted alkyl, a halogen (particularly F, CI, or Br), NO₂, or CN or X₂ and X₃ together form —S—S— and X₁ and X₄ are independently selected from H, a C₁-C₆ substituted or unsubstituted alkyl, a halogen (particularly F, CI, or Br), NO₂, or CN;
wherein optional substitution of alkyl groups is substitution with one or more halogens, —CN, —NO₂, —C(O)R′, —COOR′, —C(O)NH₂, —NHC(O)R′, —C(O)NR′R″, —CF₃, —SO₃H, —SO₂CF3, —SO₂R′, —SO₂NR′R″, —OR′, —OC(O)R′, substituted or unsubstituted phenyl, substituted or unsubstituted benzyl, substituted or unsubstituted vinyl, —NHR′ or —NR′R″, wherein R′ and R″ are independently H, an unsubstituted C₁ to C₆ alkyl or a C₁-C₃ halogen-substituted C₁-C₆ alkyl.

In embodiments, optional substitution of alkyl groups is substitution with one or more halogens, —CN, —NO₂, —CF₃, —SO₃H, or —SO₂CF₃. In embodiments, optional substitution of alkyl groups is substitution with one or more halogens.

In embodiments, R₁ and R₂ are independently unsubstituted C₁ to C₁₈ linear or branched alkyl. In embodiments, R₁ and R₂ are independently unsubstituted C₃ to C₉ linear or branched alkyl. In embodiments, R₁ and R₂ are independently unsubstituted C₃ to C₉ branched alkyl. In embodiments, R₁ and R₂ are the same group. In embodiments, R₁ and R₂ are different groups. In embodiments, R₁ and R₂ are both —CH(C₂H₅)₂.

In specific embodiments, the film formed is solvent-resistant. In specific embodiments, the film-precursor solution and the film formed are thus useful in methods of orthogonal processing. In embodiments, the film formed is resistant to water, a C₁-C₈ alcohol, a chlorinated alkane, a hydrocarbon, an aromatic hydrocarbon or an amide. In embodiments, the film formed is resistant to water, a C₁-C₆ alcohol, dichloromethane, chloroform, hexanes, xylene, benzene, toluene, or dimethylformamide.

In embodiments of Formula I, only one of X₁-X₄ is a moiety other than H. In embodiments of Formula I, only two of X₁-X₄ are moieties other than H. In specific embodiments, in the compound of Formula I all of X₁-X₄ are H. In specific embodiments, in the compound of Formula I, X₁ and X₄ are H. In specific embodiments, in the compound of Formula I, one of X₁-X₄ is a halogen, —CN or a substituted or unsubstituted C₁-C₃ alkyl group and the remaining X₁-X₄ are hydrogens. In specific embodiments of Formula I, one of X₁-X₄ is an unsubstituted C₁-C₃ alkyl. In specific embodiments of Formula I, one of X₁-X₄ is F, Br, or CI. In specific embodiments of Formula I, X₁ and X₄ are both H and X₂ and X₃ together form —S—S—. In specific embodiments of Formula I, X₁ and X₄ are both H and X₂ and X₃ are both —NO₂. In specific embodiments of Formula I, X₁, X₃ and X₄ are each H and X₂ is —CN or —NO₂.

In specific embodiments, in the compound of Formula I, R₁ and R₂ are the same group and are both selected from unsubstituted linear or branched C₃-C₁₃ alkyl groups. In specific embodiments, in the compound of Formula I, R₁ and R₂ are the same group and are both selected from unsubstituted branched C₃-C₁₃ alkyl groups. In specific embodiments, in the compound of Formula I, R₁ and R₂ are the same group and are both selected from unsubstituted linear or branched C₃-C₉ alkyl groups. In specific embodiments, in the compound of Formula I, R₁ and R₂ are the same group and are both selected from unsubstituted branched C₃-C₉ alkyl groups.

In general, any base can be used that is soluble in the solvent used for processing the film (or for making an ink) and which can deprotonate the selected N-annulated PDI compound to dissolve it in the solvent. The amount of base added to the solvent is the amount that is at least sufficient to deprotonate and dissolve the amount of N-annulated PDI compound in the solvent. In specific embodiments, the amount of base added is at least one equivalent with respect to the number of pyrrole N-H bonds in the N-annulated PDI compound in the solvent. In specific embodiments, the amount of base added is at least one equivalent ±10% with respect to the number of pyrrole N—H bonds in the N-annulated PDI compound in the solvent. In specific embodiments, the amount of base added is one equivalent ±10% with respect to the number of pyrrole N—H bonds in the N-annulated PDI compound in the solvent. In embodiments, the amount of base added is 1-5 equivalents ±10% with respect to the number of pyrrole N—H bonds in the N-annulated PDI compound in the solvent. In embodiments, the amount of base added is 2-5 equivalents ±10% with respect to the number of pyrrole N—H bonds in the N-annulated PDI compound in the solvent.

In embodiments, the base is an alkali metal hydroxide, an alkaline earth metal hydroxide or an ammonium hydroxide. In embodiments, the base is NaOH or KOH. In embodiments, the base is LiOH or CsOH. In embodiments, the base is ammonium hydroxide or an alkyl ammonium hydroxide, (R)₄N⁺OH^{—}, where each R is independently H or a C₁-C₆ alkyl group. In specific embodiments, the base is H₄NOH. In specific embodiments, the base is a tetraalkylammonium hydroxide, (R)₄N⁺OH^{—}, where each R is independently an alkyl group, particularly a C₁-C₆ alkyl group. In embodiments, the base is an alkyl amine. In embodiments, the base is an alkyl amine that is soluble in the solvent. In embodiments, the base is a primary C₁-C₈ alkyl amine. In embodiments, the base is n-butyl amine. In embodiments, the alkyl amine base can be in the form of a liquid added to the solvent or in the form of a gas under ambient conditions which can be added to the solution, for example, by bubbling the gas into the solvent until dissolution of the NPDI is achieved. In embodiments, the base is an alkali metal carbonate (e.g., sodium carbonate, potassium carbonate or cesium carbonate), an alkaline earth metal carbonate or an ammonium carbonate. In embodiments, the base is cesium carbonate. In embodiments, the base is ammonium carbonate or an alkyl ammonium carbonate, ((R)₄N⁺)₂CO₃⁻², where each R is independently H or a C₁-C₆ alkyl group. In specific embodiments, the base is a tetraalkylammonium carbonate, ((R)₄N⁺)₂CO₃⁻², where each R is independently an alkyl group, particularly a C₁-C₆alkyl group.

In specific embodiments, the solvent is a C₂-C₆ alcohol. More specifically the solvent is 1-propanol. In other embodiments, the solvent is water. In yet other embodiments, the solvent is a miscible mixture of water and a C₁-C₆alcohol.

Typically, the amount of N-annulated PDI added to the solvent is adjusted to achieve a desired film thickness. In specific embodiments, the concentration of N-annulated PDI compound in the solvent ranges from 0.1-100 mg/mL. In specific embodiments, the concentration of N-annulated PDI compound in the solvent ranges from 1-100 mg/mL. In embodiments, the concentration of NPDI in the solvent ranges from 5-50 mg/mL.

In general, any known method for forming a film from an aqueous or alcoholic solution can be employed. In embodiments, the film is formed by spin-coating. In embodiments, the film is formed by slot-die coating.

In a specific embodiment, the solution is an ink and the film is formed by printing. In general, the amount of NPDI added to a solvent to form ink depends on a given application and the printing method employed. In specific embodiments, the amount of NPDI added to form an ink is the amount necessary to form a detectible image on printing. In specific embodiments, the amount of NPDI added to form an ink is the amount necessary to form a visually detectible image on printing. In embodiments, the concentration of NPDI compound in an ink ranges from 0.1 to 100 mg/mL. In embodiments, the concentration of NPDI compound in an ink ranges from 0.1 to 50 mg/mL. In embodiments, the concentration of NPDI compound in an ink ranges from 0.1 to 10 mg/mL, 1 to 10 mg/mL, 0.1 to 20 mg/mL, 1 to 20 mg/mL, 0.1 to 5 mg/mL or 1 to 5 mg/mL.

In general, the film can be formed on any substrate that is compatible with the solvents employed. In embodiments, the film is formed on a substrate known to be appropriate for manufacture of OPV devices. In specific embodiments, the substrate is plastic, glass, quartz, foil, wood, paper or textile. In specific embodiments, the substrate is polyethylene terephthalate (PET).

In embodiments, the method of the invention for forming a film involves preparation of a film-precursor formulation which is an aqueous and/or alcoholic solution of an N-annulated PDI having at least one pyrrole N—H bond. The film-precursor formulation is formed as described herein by dissolving the N-annulated PDI compound in the selected solvent by addition of base. In embodiments, the film-precursor comprises the dissolved N-PDI compound, and base in an amount at least sufficient to dissolve the N-PDI compound in selected solvent as described above. The film-precursor formulation may comprise one or more other component useful for film formation or for printing (when the formulation is an ink).

The films and film-precursor formulations (including inks) of the invention and particularly films that are solvent-resistant are useful in the manufacture of solar cells, light emitting diodes, sensors, transistors, electrochromics, thermoelectrics, photoanodes and photocathodes among other devices. In embodiments, the solvent-resistantfilms of this invention are useful in the preparation of multi-layer devices (e.g., OPV and OLED) by orthogonal processing. More specifically, the solvent-resistant films of this invention can be employed as interface layers in such devices.

The films of the invention and more generally NPDI solids containing at least one pyrrole N—H bond can be employed to detect the presence of amines, such as an alkyl amine in liquids (e.g., in solution) and in the vapor phase. A film prepared by the methods herein wherein NPDI molecules contain the pyrrole N—H bond and which are reddish orange or red are deprotonated by contact with amines to form the corresponding anion and as a result the film changes color toward purple. The film changes color on contact with a liquid or vapor containing the amine and this color change is indicative of the presence of the amine in the liquid or vapor. An exemplary color change on contact of an NPDI film herein with an amine is illustrated in FIG. 11A. Thus, films prepared by the methods herein can be employed in colorimetric methods for the detection of amines. In related embodiments, the invention provides colorimetric detectors, i.e., sensors for amines which comprise an NPDI film as prepared herein. In embodiments, such sensors may employ visual detection of a color change (by eye). In embodiments, such sensors may be employed in combination with spectrophotometric detection methods which can detect and or quantitate a change in absorption of UV-vis light at one or more given wavelengths to detect a change in color of the film and thereby to detect the presence of an amine in contact with the film and further to quantitate the amount of amine in contact with the film. Films useful in such detectors are preferably solvent resistant to allow stable and reproducible detection of amines in solvents.

In embodiments, the invention provides a method for detecting an amine which comprises contacting a film of the invention or a film prepared by a method of the invention with a gas, liquid phase or solution which may contain an amine and detecting a color change in the film indicative of the presence of the amine in contact with the film. In embodiments, detecting a color change is visual detection. In embodiments, detecting a color change employs UV-vis spectroscopy detecting a change in absorption at one or more selected wavelengths.

The films and film-precursor formulations (including inks) of the disclosure and particularly films that are solvent-resistant are useful in the manufacture of OPV devices, particularly as an electron transporting layer in such devices. In specific embodiments, the films and film-precursor solutions (including inks) herein are useful for preparation of one or more electron transporting layer (ETL, also designated EEL) which is/are sandwiched between a bottom cathode and photoactive layer (inverted OPV device) or photoactive layer and top cathode (conventional OPV device). The ETL facilitates the transfer of electrons from the photoactive layer to the cathode in an OPV device.

The invention additionally provides an OPV device which comprises a film comprising one or more N-annulated PDI compounds. In specific embodiments, the NPDI film is employed as an ETL in an OPV device. In embodiments, the NPDI film ETL is positioned between a photoactive layer and a cathode layer in the OPV device. In related embodiments, the invention provides a method of constructing an OPV device containing one or more ETL, wherein the ETL layer is an NPDI film. In embodiments, the ETL is an NDPI film formed from an aqueous or alcoholic solution as described herein. In other related embodiments, the invention provides a method for operating an OPV device containing one or more ETL, wherein the ETL layer is an NPDI film.

The invention further provides an organic film processed via a coating or printing method using the film-precursor or ink formulation of the invention. In specific embodiments, the organic films of the invention are electron-conducting. In specific embodiments, the solvent-resistant organic films of the disclosure are electron-conducting. In related embodiments, the invention provides a method of printing which employs an aqueous or alcoholic solution of one or more NDPI compounds.

The invention further provides a film-precursor formulation which comprises one or more pyrrole-deprotonated N-annulated PDI compound in a solvent selected from water, a C₁-C₆alcohol or a miscible mixture thereof. In embodiments, the formulation comprises a deprotonated N-annulated PDI of Formula I. In embodiments, the formulation is a solution in a C₂-C₆ alcohol. In embodiments, the formulation is an aqueous solution. In embodiments, the formulation is a solution in water.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1: Chemical structure of PDIN-H (compound 1) during film formation. PDIN-H is an insoluble red dye. Treatment of a 10 mg/mL slurry of PDIN-H in 1-propranol with 1 molar equivalent of NaOH produces a purple ionic solution, containing the soluble purple dye PDIN⁻Na+. Spin-coating this solution onto a substrate (e.g., PET) yields a red colored organic film believed to be due to the spontaneous protonation of the PDIN anion. This processes mimics that of classic acid dyeing of textiles.

FIG. 2: A photograph of mixtures of 5 mg/mL PDIN-H in various solvents to demonstrate the low solubility of the PDI-NH compound itself in (Left to Right): 1. 1-propanol (orange solution with precipitated solid), 2. Dichloromethane (orange solution with precipitated solid), 3. o-xylene (orange solution with precipitated/suspended solid), 4. Chloroform (reddish orange suspension of solid), 5. dimethyl formamide (reddish orange suspension), and 6. Hexanes (solid precipitated in clear solvent).

FIGS. 3A and 3B: FIG. 3A) Photograph showing the color changes of PDIN-H in 1-propanol solution with varying equivalents of NaOH added, from 0.5 to 10 eq., as indicated. At 0.5 eq. the solution is yellow and the color of the solution deepens on addition of base to orange at 2.0 eq. The solution is reddish purple at 3.0 eq. changing to a deeper purple through 10 eq. FIG. 3B) UV-vis spectra of PDIN-H solutions in CHCI₃ alone (0) and with 1 mol equivalent NaOH (0+), and in 1-propanol with 0-10 mol equivalents of NaOH (1-8) exhibits the color changes noted in FIG. 3A.

FIGS. 4A and 4B: FIG. 4A) Images of spin-coated 1.5 cm ×1.5 cm films at top (T). films are reddish-orange. Images of slot-die coated 13 mm × 20 cm film at bottom (B). (1) ethanol, (2) 2-propanol, (3) 1-propanol, (4) 1-butanol, (5) 1-pentanol, (6) 1-hexanol. Films prepared from 10 mg/mL solutions of PDIN-H in the indicated alcohol with 1 molar equivalent of NaOH added. Films are reddish-orange with some variation in depth of color with solvent used. FIG. 4 B) Images of slot-die coated films from 1-propanol solutions with varying PDIN-H concentrations: (7) 5 mg/mL, (8) 10 mg/mL, (9) 20 mg/mL, (10) 30 mg/mL, (11) 40 mg/mL, (12) 50 mg/mL. In all solutions 1 molar equivalent of NaOH was added. Films range in color from reddish orange to deeper red as concentration of PDIN-H increased.

FIGS. 5A-C: (FIG. 5A) Optical absorption spectra of PDIN-H films spin-coated from indicated alcohols at 10 mg/mL, absorption intensity generally increases from 1-hexanol (6), to 1-pentanol (5), to 1-butanol (4), to ethanol (3), and 1-propanol (2), to 2-propanol (1). (FIG. 5B) Optical absorption spectra of PDIN-H films slot-die coated from indicated alcohols at 10 mg/mL, and (FIG. 5C) Optical absorption spectra of PDIN-H films slot-die coated from 1-propranol at varying concentrations, as indicated, onto PET. In all cases 1 molar equivalent NaOH was added to each pre-deposition solution. Absorbance generally increases with concentration (1-6).

FIG. 6: Illustration of solvent resistance of films. Photographs of 1.5 cm x 1.5 cm (top), optical microscopy images (10×magnification with white scale bar corresponding to 500 µm) (middle), and atomic force microscopy height images (bottom) for PDIN-H films on PET: I) as-cast with no solvent coated on top and with II) 2-propanol, III) water, or IV) o-xylene slot-die coated on top and left to dry. PDIN-H films formed via slot-die coating 10 mg/mL PDIN-H solutions in 1-propanol with 1 molar equivalent of NaOH added. For the atomic force microscopy height images on the bottom of the figure, the color range from dark to light ranges from 0.0, 10.0, 15.0 to 22.9 nm for I (with no solvent treatment), from 0.0, 10.0, 15.0, 20.0, 25.0 to 29.3 nm for II (treatment with 2-propanol), from 0.0, 10.5, 15.0 to 27.3 nm for III (treatment with water), and ranges from 0.0, 5.0, 10.0, 15.0 to 18.5 nm for IV (treatment with o-xylene)

FIGS. 7A and 7B: Illustration of solvent resistance. (FIG. 7A) Optical absorption spectra of PDIN-H films as-cast with no solvent (0) coated on top and with 2-propanol (1), water (2), or o-xylene (3) slot-die coated on top of film and left to dry. A minor shift in spectrum is observed on treatment with o-xylene. (FIG. 7B) PDIN-H films as-cast (0) or dipped in 2-propanol (1), water (2), or o-xylene (3) for 10 s then removed and left to dry.

FIG. 8: Illustration of solvent resistance. Photographs (1.5 cm ×1.5 cm) for PDIN-H slot-die coated films dipped in various solvents for 10 s and allowed to air dry: no solvent, water, 2-propanol and o-xylene (Left to Right). Films were formed by slot-die coating a solution of PDIN-H in 1-propanol (10 mg/mL) with 1 molar equivalent of NaOH added. The films are reddish orange in color with intensity decreasing somewhat from water, to 2-propanol to o-xylene.

FIGS. 9A-9C: Images created from single-crystal XRD data. (FIG. 9A) Packing diagram showing distinct hydrogen bonding between the NH and CO functional groups. (FIG. 9B) Packing diagram showing tight π-stacks. (FIG. 9C) Packing diagram showing the offset and dihedral angle between two π-stacked molecules of PDIN-H.

FIG. 10: UV-vis spectra of slot-die coated PDIN-H from 1-propanol (10 mg/mL) with 1 molar equivalent of NaOH before (0) and after (1) exposure to butylamine vapor for 1 minute (1 mL of butylamine was deposited at the bottom of a closed container and the film was deposited on a watch glass).

FIGS. 11A-11C: (FIG. 11A) Chemical structure of PDIN-H and photographs of corresponding slot-die coated films (1) as-cast, (2) during exposure to butylamine vapor, and (3) after removal of butylamine vapor. As-cast films are reddish-orange. On exposure to butylamine vapor, the film changes color to purple. Purple film returns to its original reddish orange color on removal of butylamine vapor. The PDIN-H films were processed from 10 mg/mL 1-propanol solutions with 1 molar equivalent NaOH added. (FIG. 11B) Top: PDIN-H in 1-propanol (10 mg/mL) with indicated added volume equivalents of butylamine. All samples have a liquid phase above a dark solid. As butylamine is added, the liquid phase above the solid becomes pinkish compared to no butylamine control and increase in intensity to a reddish purple as more butylamine is added. Bottom: PDIN-H in water (10 mg/mL) with indicated added volume equivalents of butylamine. All samples have a liquid phase above a dark solid. On addition of butylamine, the liquid phase becomes light purple which increases in intensity as more butylamine is added. (FIG. 11C) Photographs of PDIN-H films prepared via slot-die coating from: Top: 1-propanol/butylamine (1:1 v/v) solutions of PDIN-H (5 mg/mL). Bottom: water/butylamine (1:1 v/v) solutions of PDIN-H (5 mg/mL). Both films are reddish orange in color with the bottom film prepared from water solution somewhat more intense in color.

FIG. 12A: Top - two photographs (1.5 cm ×1.5 cm) of films- (Left) spin-coated PDIN-H in 1-propanol (10 mg/mL) with added volume equivalents of butylamine on PET - (Right) slot-die coated PDIN-H in 1-propanol (10 mg/mL) with added volume equivalents of butylamine on PET. Bottom - below the photographs are the UV-vis spectra of the films of the photographs. Both films are reddish orange with the spin-coated film (Left) having somewhat increased intensity.

FIG. 12B: Top - two photographs (1.5 cm×1.5 cm) of films- (Left) spin-coated PDIN-H in water (10 mg/mL) with added volume equivalents of butylamine on PET - (Right) slot-die coated PDIN-H in water (10 mg/mL) with added volume equivalents of butylamine on PET. Bottom - below the photographs are the UV-vis spectra of the films of the photographs. Both films are reddish orange in color.

FIG. 13A: Top - four photographs (1.5 cm×15.cm) of PDIN-H films formed via slot-die coating 10 mg/mL PDIN-H solutions in 1-propanolwith 1 volumetric equivalent of butylamine added. (Photographs Left to Right) as-cast with no solvent coated on top; cast with 2-propanol, water, or o-xylene slot-die coated on top of film and left to dry. Bottom - below the photographs are the UV-vis spectra of the films of the photographs.

FIG. 13B: Top - four photographs (1.5 cm ×15.cm) of PDIN-H films formed via slot-die coating 10 mg/mL PDIN-H solutions in water with 1 volumetric equivalent of butylamine added. (Left to Right) as-cast with no solvent coated on top; cast with 2-propanol, water, or o-xylene slot-die coated on top and left to dry. Bottom - below the photographs are the UV-vis spectra of the films of the photographs.

FIG. 14 illustrates UV-vis spectra of PDIN-H films formed by slot-die roll coating of 1-propanol solutions (5 mg/mL) containing 1 molar equivalent of T tetramethylammonium hydroxide (TMAOH), tetraethylammonium hydroxide (TEAOH), tetra(n-propyl)ammonium hydroxide (TPAOH), or tetra(n-butyl)ammonium hydroxide (TBAOH). The UV-vis spectra of the films were similar. Films exhibited solvent resistance to water, 2-propanol and somewhat less consistently to o-xylene.

FIGS. 15A and 15B illustrate solvent-resistant NPDI films formed using cesium carbonate as the base. FIG. 15A is a photograph of a series of solutions of PDIN-H in 1-propanol (0.5 mg/mL) with additions of 0.5 eq. to 10 eq. of cesium carbonate. The color of the solution on addition of 0.5 eq. is yellow. As additional equivalents of base are added the solution becomes orange/reddish orange by about 2 eqs. As additional equivalents of the base are added the color of the solution changes to reddish purple and ultimately to purple. Purple solutions are indicative of the presence of the NPDI anion. FIG. 15 B illustrates the change in UV-vis spectrum of films prepared with 1-propanol solutions of PDIN (0.5 mg/mL) with varying equivalents of cesium carbonate added.

FIGS. 16A-16D illustrate exemplary OPV device construction and properties. FIG. 16A illustrates OPV device structure where ETL is ZnO, ZnO/PDNI-H or PDIN-H, where the PDIN-H layer is processed from PDIN-H solutions in 1-propanol with 1 molar equivalent of NaOH added. FIG. 16B presents EQE spectra of devices of the structure of FIG. 16A with indicated ELT. FIGS. 16C and 16D present graphs of current density-voltage characteristics under 1-sum illumination (C) and in the dark (D).

### DETAILED DESCRIPTION

The invention relates to organic semiconductor thin films which are useful, for example, in electronic devices. The disclosure relates to thin films comprising one or more N-annulated PDI compounds, methods of making films, and particularly to film processing methods that employ green solvents, such as water, aqueous solutions and alcohols. The disclosure also relates to films made by the methods herein and to film-precursor formulations and inks which are solutions in which one or more N-annulated PDI compounds are solubilized and from which films can be prepared.

More specifically, the disclosure provides a thin film comprising one or more an N-annulated perylene diimide (PDI) compounds having a pyrrolic N—H bond and methods for making such films using green solvents.

In embodiments, the N-annulated PDI compound is a compound of Formula I:

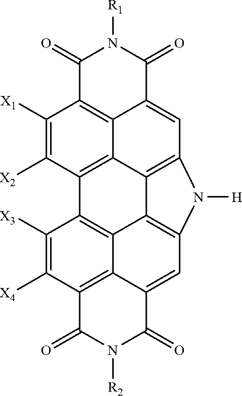

wherein:
R₁ and R₂ are independently a substituted or unsubstituted C₁ to C₁₈ linear or branched alkyl; and
X₁-X₄ are independently selected from H, a C₁-C₆ substituted or unsubstituted alkyl, a halogen (particularly F, CI, or Br), NO₂, or CN or X₂ and X₃ together form —S—S— and X₁ and X₄ are independently selected from H, a C₁-C₆ substituted or unsubstituted alkyl, a halogen (particularly F, CI, or Br), NO₂, or CN;
wherein optional substitution of alkyl groups is substitution with one or more halogens, —CN, —NO₂, —C(O)R′, —COOR′, —C(O)NH₂, —NHC(O)R′, —C(O)NR′R″, —CF₃, —SO₃H, —SO₂CF3, —SO₂R′, —SO₂NR′R″, —OR′, —OC(O)R′, substituted or unsubstituted phenyl, substituted or unsubstituted benzyl, substituted or unsubstituted vinyl, —NHR′ or —NR′R″, wherein R′ and R″ are independently H, an unsubstituted C₁ to C₆ alkyl or a C₁-C₃ halogen-substituted C₁-C₆ alkyl.

In specific embodiments, alkyl substitution is substitution with one or more halogens, —CN, —NO₂, —CF₃, —SO₃H, or —SO₂CF₃. In embodiments, optional substitution of alkyl groups is substitution with one or more halogens.

In embodiments, R₁ and R₂ are independently unsubstituted C₁ to C₁₈ linear or branched alkyl. In embodiments, R₁ and R₂ are independently unsubstituted C₃ to C₉ branched alkyl. In embodiments, R₁ and R₂ are the same group. In embodiments, R₁ and R₂ are different groups. In embodiments, R₁ and R₂ are the same alkyl group. In embodiments, R₁ and R₂ are different alkyl groups.

In embodiments, all of X₁-X₄ are hydrogen. In embodiments, one of X₁-X₄ is a non-hydrogen group as listed above. In embodiments, X₁, X₃ and X₄ are hydrogens and X₂ is a non-hydrogen substituent as listed above. In embodiments, X₂, X₃ and X₄ are hydrogens and X₁ is a non-hydrogen substituent as listed above. In embodiments, one of X₁-X₄ is a halogen. In embodiments, one of X₁-X₄ is —CN. In embodiments, one of X₁-X₄ is a C₁-C₃ unsubstituted alkyl. In embodiments, X₂ or X₁ is selected from a halogen, —CN or an unsubstituted C₁-C₃ alkyl.

In embodiments, X₁-X₄ are independently selected from H, a C₁-C₆ substituted or unsubstituted alkyl, F, CI, Br, NO₂, or CN and only two of X₁-X₄ are moieties other than H. In embodiments, X₁-X₄ are independently selected from H, a C₁-C₆ substituted or unsubstituted alkyl, F, CI, Br, NO₂, or CN and only one ofX₁-X₄ is a moiety other than H.

In embodiments, the N-annulated PDI compound is compound 1:

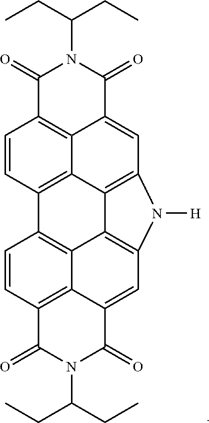

In embodiments, the N-annulated PDI compound is a compound of Formula I, wherein X₁ and X₄ are both H, R₁ and R₂ are both unsubstituted alkyl groups having 3 to 8 carbon atoms, and X₂ and X₃ are independently selected from H, —CN, —NO₂, or halogen or X₂ and X₃ together form —S—S—. In further embodiments, one of X₂ or X₃ is —CN, —NO₂ or halogen. In further embodiments, one of X₂ or X₃ is Br or CI. In further embodiments, R₁ and R₂ are branched alkyl groups having 3 to 8 carbon atoms. In further embodiments, R₁ and R₂ are both —CH(C₂H₅)₂.

In an embodiment, the thin film is solvent-resistant. In embodiments, the film is resistant to water, a C₁-C₈ alcohol, a chlorinated alkane, a hydrocarbon, an aromatic hydrocarbon or an amide. In embodiments, the thin film is resistant to water, a C₁-C₆ alcohol, dichloromethane, chloroform, hexanes, xylene, benzene, toluene, or dimethylformamide. Resistance to a given solvent can be assessed by noting or measuring changes in film morphology or film properties on contact of the film with a given solvent. In particular, a solvent-resistant film is not measurably dissolved or dissociated on contract with a solvent to which it is resistant. It is noted that films of different N-annulated PDI compounds may be resistant to different solvents. Solvent-resistant films are of particular interest for orthogonal processing methods for the construction of layered electronic devices. A solvent-resistant film can be successfully employed in such methods where additional layers are formed upon and in contact with the solvent-resistant without measurable detriment to the solvent-resistant film

In embodiments, the thin film ranges in thickness from 10 to 1000 nm thick. In embodiments, the film ranges in thickness from 100 to 1000 nm thick. In embodiments, the thin film ranges in thickness from 10 to 500 nm. In embodiments, the film is uniform, such that no feature of the film is greater than 1000 nm in any dimension. In embodiments, no feature of the film is greater than 500 nm in any dimension. In embodiments, no feature of the film is greater than 250 nm in any dimension.

In embodiments, the thin film is formed on a substrate. In generally, the film can be formed on any substrate including substrates known to be useful in the fabrication of layered electronic devices. In embodiments, the film is formed on plastic, glass, quartz, metal foil, wood, paper or textile. In embodiments, the substrate may be flexible or rigid. In an embodiment, the film is formed on PET. In an embodiment, the thin film may be formed on and in contract with another layer of thin film, such as by orthogonal processing. In an embodiment, the film is formed by printing by any compatible printing process, for example, by ink-jet printing.

The method for forming a thin film of the disclosure involves providing a film-precursor formulation or an ink which comprises an N-annulated PDI compound having a pyrrole N—H group dissolved in an aqueous or alcohol solvent. The film-precursor formulation or ink is used to form the film by any known compatible film processing method or by any compatible printing method. In embodiments, the film consists essentially of the N-annulated PDI compound and the components of the base added to solubilize the N-annulated PDI compound in the selected green solvent. Such base components can include metal cation components of a base. This embodiment does not exclude additives such as plasticizers, surfactants and other surface active materials that are added at levels that do not affect the formation of solutions or films. In embodiments, the films consist of N-annulated PDI compound and the components of the base added to solubilize the N-annulated PDI compound in the selected green solvent. Such base components can include metal cation components of a base.

More specifically, a thin film of the disclosure is formed by first dissolving a selected amount of the N-annulated PDI compound in a selected amount of a solvent selected from water, a C₁-C₆ alcohol or a miscible combination thereof by addition to the solvent containing the N-annulated compound of an amount of base at least sufficient to polarize the pyrrole N—H bond giving an ionic salt dissolved in the solvent. The amount of N-annulated PDI compound dissolved in solution is that amount needed to form a film of desired thickness in a selected amount of solution. The amount of N-annulated PDI needed in the solution for formation of a film of selected thickness can be determined by routine experimentation. The amount of base added to the formulation is an amount sufficient to dissolve the N-annulated PDI compound. Again, the amount of bases that is needed to dissolve a selected amount of N-annulated PDI compound can be determined in a selected solvent by routine experimentation. In an embodiment, the amount of base added is about one equivalent (±10%) with respect to the number of pyrrole N—H bonds in the N-annulated PDI compound, which is typically one. In an embodiment, the amount of base added to dissolve the N-annulated PDI compound is at least one equivalent with respect to pyrrole N—H bonds in the N-annulated PDI compound. In embodiments, more than one equivalent of base can be added. In embodiments, two or more equivalents of base can be added.

After preparation of the film-precursor formulation by dissolving the N-annulated PDI compound in selected solvent, a film is formed using any compatible method. A thin film, particularly, a thin film patterned on a substrate can be formed by printing. The final dried film is formed by removing solvent from the film. In an embodiment, removing solvent from the film as coated, applied or printed results in a solvent-resistant film.

After dissolution of the N-annulated PDI compound in solvent, the solution is optionally filtered to remove particles of a selected high limit of particle size. In embodiments, particles of size of 1 nm or greater are removed. In an embodiment, particle of size greater than 0.5 nm are removed.

In embodiments, the film is formed by spin-coating. In embodiments, the film is formed by slot-die coating. In embodiments, a film is formed by printing. Concentrations of NPDI compound and base are adjusted as known in the art for a given film thickness and film-forming or printing method.

It is believed that addition of base to a slurry of N-annulated PDI compound in aqueous or alcoholic solvent results in ionization of the N-annulated PDI compound to form the deprotonated anionic species in solution.

In embodiments, the concentration of N-annulated PDI compound in the solvent ranges from 0.1 to 100 mg/mL In embodiments, the concentration of N-annulated PDI compound in the solvent ranges from 1 to 100 mg/mL In embodiments, the concentration of N-annulated PDI in the solvent ranges from 0.5-50 mg/mL. In embodiments, the concentration of N-annulated PDI in the solvent ranges from 5-50 mg/mL. In embodiments, the concentration of N-annulated PDI in the solvent ranges from 10-40 mg/mL. In embodiments, the concentration of N-annulated PDI in the solvent ranges from 0.1-10 mg/mL In embodiments, the concentration of N-annulated PDI in the solvent ranges from 1-10 mg/mL

In an embodiment, the thin film is a solvent-resistant, organic semiconducting film comprising non-polymeric perylene diimide molecules with pyrrolic N—H bonds. In embodiments, the solvent-resistant film comprises an N-annulated PDI compound of Formula I.

The term alkyl refers to a monovalent group formally derived from a saturated hydrocarbon group by removal of a hydrogen. An alkyl group has the general formula CₙH₂ₙ₊₁. Alkyl groups can be straight-chain (linear) or branched. Alkyl groups herein can have 1-18 carbon atoms and more preferably 3-13 carbon atoms. Branched alkyl groups herein can have 3-30 carbon atoms and more preferably 3-18 carbon atoms. Straight-chain alkyl groups include those having 1-3 carbon atoms, 1-6 carbon atoms, 1-12 carbon atoms, 1-18 carbon atoms, 4-8 carbon atoms, 6-12 carbon atoms, and 4-12 carbon atoms, among other groups of carbon atom range. Carbon atom range in alkyl groups herein is expresses as a Cx to Cy alkyl group, where x and y are integers representing the number of carbons in the group. Straight-chain alkyl groups include methyl, ethyl, n-propyl, n-hexyl, n-heptyl, etc. individually or in any combination. Branched alkyl groups include iso-propyl, isobutyl, sec-butyl, 1-ethylpropyl, 1-propylbutyl, 1-butylpentyl, 1-pentylhexyl, 1-hexylheptyl, 1-heptyloctyl, 1-octylnonyl, 1-nonyldecyl, 2-ethylhexyl individually or in any combination. Branching may occur anywhere along the alkyl chain from the site of attachment of the alkyl group. For example, a branch may occur at the first carbon (as in a 1-ethylpropyl group). The branching can occur for example at the second carbon along the chain (e.g., 2-ethylhexyl). There may be multiple branches along the chain (e.g., 1-ethyl-5-methylhexyl). In specific embodiments, a branched alkyl chain has one branching point which is at the first, second or third carbon from the site of attachment. Alkyl groups herein are optionally substituted. When an alkyl group is described as a Cx to Cy alkyl compound, this encompasses all alkyl groups having x to y carbon atoms including all isomers thereof.

Herein cycloalkyl is a subset of alkyl groups having a carbon ring of 3 or more atoms, typically 3-12 carbon atoms, 3-6 carbon atoms or 3-10 carbon atoms. In specific embodiments, cycloalkyl groups have 3, 4, 5, 6, 7 or 8 member carbon rings. Herein a cycloalkyl can replace an alkyl group. Specific cycloalkyl groups include cyclopropyl, cyclobutyl, cyclopentyl, cyclohexyl, cycloheptyl, cyclooctyl, cyclononyl and cyclodecyl groups. Cycloalkyl groups are optionally substituted. Where a substituent is designated an alkyl group that designation includes cycloalkyl groups.

As described herein alkyl groups are optionally substituted with one or more non-hydrogen groups selected from wherein optional substitution of alkyl groups is substitution with one or more halogens, —CN, —NO₂, —C(O)R′, —COOR′, —C(O)NH₂, —NHC(O)R′, —C(O)NR′R″, —CF₃, —SO₃H, —SO₂CF3, —SO₂R′, —SO₂NR′R″, —OR′, —OC(O)R′, substituted or unsubstituted phenyl, substituted or unsubstituted benzyl, substituted or unsubstituted vinyl, —NHR′ or —NR′R″, wherein R′ and R″ are independently H, an unsubstituted C₁ to C₆ alkyl or a C₁-C₃ halogen-substituted C₁-C₆ alkyl. More preferred substituents are halogen, —CN, —NO₂, —CF₃, —SO₃H, —SO₂CF₃, or —SO₂R′. Yet more preferred substituents are F, CI, Br, or —CN. In embodiments, alkyl groups carry one of the listed substituents. In embodiments, alkyl groups carry two of the listed substituents. In embodiments, alkyl groups carry three of the listed substituents. In embodiments, alkyl groups are unsubstituted.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

With respect to the various compounds of the disclosure, the atoms therein may have various isotopic forms (e.g., isotopes of hydrogen include deuterium and tritium). All isotopic variants of compounds of the disclosure are included within the disclosure and particularly include deuterium and ¹³C isotopic variants. It will be appreciated that such isotopic variants may be useful for carrying out various chemical and biological analyses, investigations of reaction mechanisms and the like. Methods for making isotopic variants are known in the art.

Compounds of the disclosure can be prepared by one of ordinary skill in the art in view of the descriptions provided herein and what is known in the art from commercially or otherwise readily available starting materials and reagents. As described herein in the Examples, known synthetic methods can be readily adapted for synthesis of the compounds of the formulas herein.

In embodiments, the N-annulated PDI compounds herein are not soluble in alcohol or water at a sufficiently high amount to allow formation of a film of desired thickness and concentration of NPDI compound from the selected solvent. Films herein may be prepared from solutions containing more than one NPDI compound and containing one base or a mixture of bases.

Base is added to formulations herein to facilitate dissolution of N-annulated PDI compounds in the formulation. Any base that can function to deprotonate the pyrrolic N—H bond of the N-annulated PDI compound can be used. The base can be any source of OH^{—}. The base can be any source of carbonate. In specific embodiments, the base is an inorganic base, such as an alkali metal hydroxide or an alkaline earth metal hydroxide. In specific embodiments, the base is an alkali metal carbonate or an alkaline earth metal carbonate. In a specific embodiment, the base is cesium carbonate. The base can also be an ammonium hydroxide and more particularly an alkyl ammonium hydroxide base. The term ammonium refers to the (R)₄N⁺ cation, where each R is independently H or an alkyl group. In embodiments, the base can be a quaternary alkyl ammonium hydroxide. Preferred alkyl groups for ammonium compounds are C₁-C₆ alkyl groups and more preferred are C₁-C₄ alkyl groups. The base can also be an organic base, such as an amine. In specific embodiments, the amine is an alkyl amine, which includes a monoalkyl amine (a primary amine), dialkyl amine (a secondary amine) or a trialkyl amine (a tertiary amine). In specific embodiment, the base is a primary amine. In embodiments, the primary amine is a C₁-C₆ alkyl primary amine (NH₂R, where R is a C₁-C₆ alkyl group). The base may be a solid, liquid or a gas at ambient temperatures. A base that is a gas can be contacted with the solvent by bubbling the gas through the solvent, for example. The base is preferably soluble in the aqueous or alcoholic solvent used in the formulation at the concentration at which it is added to the formulation. In general, base is added to a given formulation in an amount that dissolves the N-annulated PDI compound. In specific embodiments, the amount of base added is at least about one equivalent (±10%) of base with respect to the number of pyrrolic N—H bonds in the N-annulated PDI compound in the solvent. In specific embodiments, the amount of base added to the formulation is one equivalent (±10%) with respect to the number of pyrrolic N—H bonds in the N-annulated PDI compound in the solvent. In specific embodiments, the amount of base added to the formulation is at least two equivalents, or at least 3 equivalents (±10%) with respect to the number of pyrrolic N—H bonds in the N-annulated PDI compound in the solvent. In specific embodiments, the amount of base added to the formulation is at least two equivalents, or at least 3 equivalents (±10%), but less than 5 equivalents (±10%), with respect to the number of pyrrolic N—H bonds in the N-annulated PDI compound in the solvent.

Solvents of the formulations, including inks, herein are most generally green solvents and include water, C₁-C₈ alcohols and miscible mixtures thereof, among others. More specifically, the solvents are water, aqueous solutions and miscible mixtures of water with an alcohol. In specific embodiments, the solvent is water, a C₂ to C₆ alcohol or a miscible mixture thereof. In specific embodiments, the solvent is n-propanol. IN specific embodiments, the solvent is water.

Film-precursor formulations herein may further comprise one or more functional additives that facilitate film formation or function. Ink formulations herein may further comprise one or more functional additives that facilitate use of the ink for printing. Formulations herein may, for example, comprise one or more surfactants, biocides, corrosion inhibitors, plasticizers, viscosity modifiers, other colorants or the like. However, the predominant components of the formulations herein are solvent and N-annulated PDI (or the anion thereof). In embodiments, combined additives in a formulation represent less than about 10% of the weight of N-annulated PDI in the formulation. In embodiments, combined additives in a formulation represent less than about 5% of the weight of N-annulated PDI in the formulation. In embodiments, combined additives in a formulation represent less than about 1% of the weight of N-annulated PDI in the formulation.

In embodiments, solutions for film formation may be filtered prior to film formation using appropriate filtration media to remove undesired particulate or other solid materials.

Any method known in the art for forming thin films which can employ aqueous or alcoholic solutions for film formation can be employed herein. In specific embodiments, films are formed by spin coating. In embodiments, films are formed by slot-die methods.

Films of this invention can be employed, for example, as an electron acceptor in electronic devices. Exemplary electronic devices include among others an organic solar cell, an organic thin film transistor, a Li-ion battery. Those of ordinary skill in the art will appreciate that methods for the preparation of organic solar cells, organic thin film transistors and Li-ion batteries and other electronic devices that employ electron acceptor films are known in the art and can be applied employing materials of the formulas herein. In view of what is known in the art and what is described herein one of ordinary skill in the art can employ materials described and characterized herein in such devices without resort to undue experimentation.

The films and film-precursor formulations of the invention, and particularly films that are solvent-resistant are useful in the manufacture of OPV devices, particularly as an electron transporting layer in such devices. In specific embodiments, the films and film-precursor solutions herein are useful for preparation of one or more electron transporting layer (ETL, also designated EEL) which is/are sandwiched between a bottom cathode and photoactive layer (inverted OPV device) or photoactive layer and top cathode (conventional OPV device). The ETL facilitates the transfer of electrons from the photoactive layer to the cathode in an OPV device.

The invention additionally provides an OPV device which comprises a film comprising one or more N-annulated PDI compounds. In specific embodiments, the NPDI film is employed as an ETL in an OPV device. In embodiments, the NPDI film ETL is positioned between a photoactive layer and a cathode layer in the OPV device. In related embodiments, the invention provides a method of constructing an OPV device containing one or more ETL, wherein the ETL layer is an NPDI film. In embodiments, the ETL is an NDPI film formed from an aqueous or alcoholic solution as described herein. In other related embodiments, the invention provides a method for operating an OPV device containing one or more ETL, wherein the ETL layer is an NPDI film.

OPV cells typically have layered structures, where a photoactive layer is sandwiched between two electrodes. A hole extraction layer (HEL) and an electron extraction layer (ETL or EEL) facilitate efficient current between the active layer and the electrodes. There are two different OPV geometries: normal and inverted, the structures of which are known in the art. In normal cells typically indium tin oxide (ITO) is used as the anode and a metal with a lower work function than ITO (e.g. aluminum) is employed as the cathode. In inverted cells, the cathode is usually ITO and the anode is a metal with a work function higher than ITO (e.g. silver). Inverted OPV modules are generally more stable and show higher efficiencies. Interfacial layers can significantly improve the function of OPV cells. [71]. NPDI films of this invention can be employed as interfacial layers in OPV devices. NPDI films of this invention can be employed in particular as ETL in OPV. Note that OPV devices may contain one or more NPDI-films as interfacial layers in OPV devices.

Additional details of the structure and manufacture of OPV devices and interfacial layers therein are provided in references cited herein, such as reference 71. Each such reference, including reference 71 are incorporated by reference herein in its entirety for descriptions of materials used in constructing OPV devices and more particularly ETL layers.

Additional details of the synthesis, characterization and application of N-annulated PDI materials and films thereof are provided in references cited herein and any supporting information of each of these references, which is freely available on-line for the publisher. Each cited reference herein, including any electronic supplemental information thereof, providing such additional description is incorporated by reference herein in its entirety for descriptions of film making techniques and methods for fabrication electronic devices incorporating such films.

Additional details of processing of materials, such as N-annulated PDI materials and films thereof of the invention, and the preparation of devices, such as organic solar cells are provided in certain references cited herein and any supporting information of each of these references which is freely available on-line for the publisher. Each of the references cited herein and any corresponding supporting information is incorporated by reference herein in its entirety for such additional details including synthetic methods for starting materials, purification methods, characterization of compounds, processing of materials, components of devices employing these materials and methods for such characterization, construction and testing of organic solar cell, as well as structure and components of organic solar cells.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

The description herein may refer to a color of a film, solution or liquid phase. When provided such color designations are based on visual observation of the item begin described or a photograph of such item by a person believed to have normal color vision. It will be appreciated that the color description given are subjective to the observer. This, designations including yellow, reddish orange, and purple among others should be considered approximations of the actual color of the item described. UV-vis spectra of films, solutions and liquid phases, which are provided in some cases herein, provide a quantitative method for assessment of the color of a given item. In visual colorimetric detection methods herein the color change indicative of the presence of amines is described as a change from reddish orange/red to purple. This color change may be described differently by different individual observers.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

One of ordinary skill in the art will appreciate that methods, process conditions, concentration, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. For compositions as claimed herein, the term consisting essentially of excludes any component that detrimentally and materially affects the properties of that composition for use in an application recited herein, such as use of the composition as an electron acceptor particularly in an electronic device or more specifically in a thin film transistor, or a Li-ion battery.

Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

### Example 1: Preparation of Solvent Resistant Films of PDIN-H Using Hydroxide Base

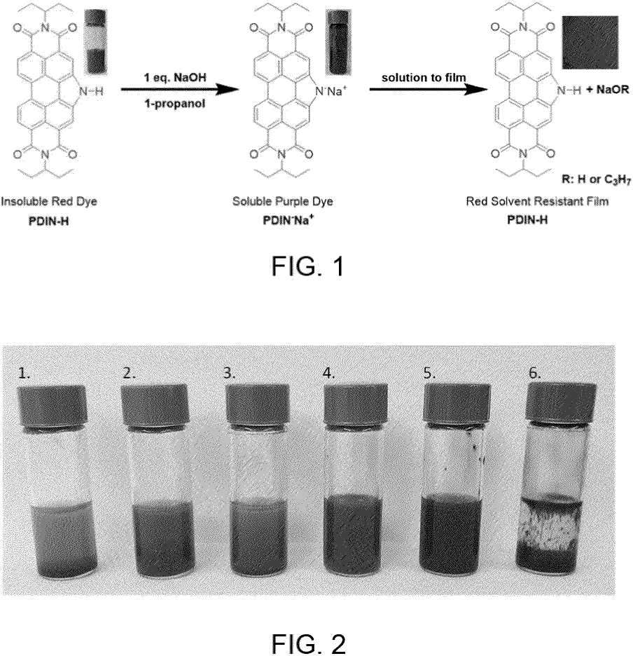

The N-annulated PDI (PDIN-H, FIG. 1) is readily synthesized and purified column chromatology free, on multi-gram scale. [22] The compound is bright red and is only sparingly soluble in halogenated solvents such as CHCI₃ or polar solvents such as isopropanol (FIG. 2). Upon addition of NaOH to a slurry of isopropanol and PDIN-H (10 mg/mL) a color change from orange/red to purple and complete dissolution of the PDIN-H is observed. Spin-coating this solution onto an anti-static coated polyethylene terephthalate (PET) substrate results in the formation of a uniform red thin-film. In this process the NaOH is believed to deprotonate the pyrrole N—H of PDIN-H creating an alcohol soluble ionic dye, PDIN⁻Na⁺. A transition from solution to film shifts back to the PDIN-H giving the distinct PDI red color (FIG. 1).

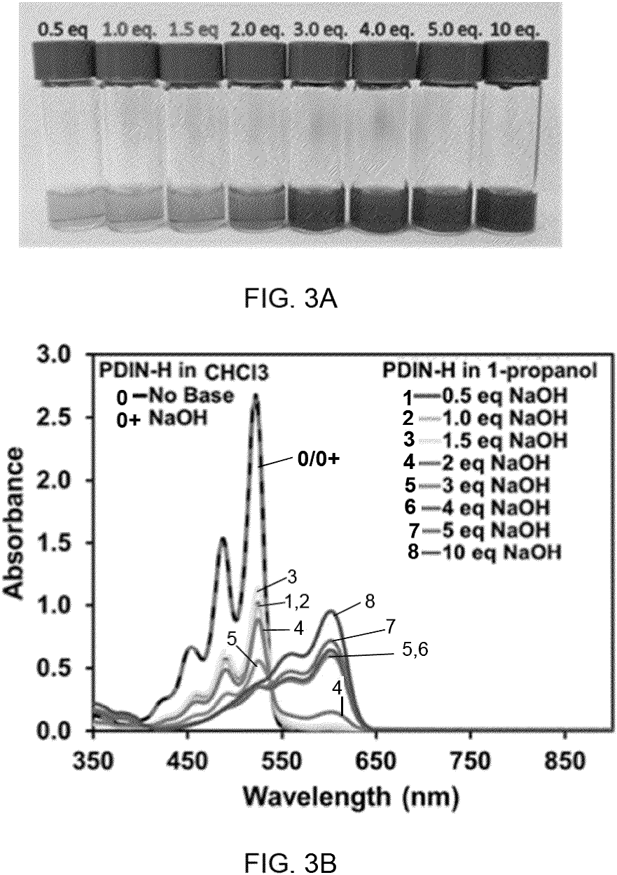

This process was further investigated in dilute solution using optical absorption spectroscopy (FIG. 3). In dilute CHCl₃ solution, PDIN-H gives rise to a typical PDI based absorption spectrum with strong absorption from 400-550 nm with three diagnostic vibronic bands. Adding base has no effect on the spectrum. For solutions of PDIN-H in 1-propanol (PDIN-H concentration of 0.01 mg/mL), a gradual change in color from orange to purple with increasing equivalents of NaOH added (up to 10 molar equivalents, with respect to PDIN-H) was observed. The gradual change in dilute solution indicates an equilibrium in favor of the PDIN-H. UV-visible spectroscopic analysis of the solutions reveals that at 0.5 eq. NaOH added, the dominate absorption profile of PDIN-H is present with characteristic PDI bands at 459, 490, and 525 nm. At 1.0 eq. NaOH added a small lower energy band at around 601 nm appears. This new band is attributed to the PDIN-anion. At 2 eq. NaOH the two species have near equal absorbance intensity. At 3 eq. NaOH and above it appears that the PDIN-H is fully deprotonated giving rise to an absorbance spectrum of only the PDIN⁻ anion, characteristics which have been observed in other PDI based molecules. [47, 48]

To expand the solvent scope for solution preparation and subsequent film formation a series of six alcohols were tested and solutions both spin-coated and slot-die coated onto PET substrates. Multiple PDIN-H/NaOH solutions were prepared at 10 mg/mL in the following alcohols: ethanol, 2-propanol, 1-propanol, 1-butanol, 1-pentanol, and 1-hexanol. Only 1 molar equivalent of NaOH was required to solubilize the PDIN-H. Methanol failed to yield solutions suitable for uniform film formation as the PDIN-H did not dissolve and thus be converted to the anion. The longer chain alcohols 1-heptanol and 1-decanol were not suitable for roll-to-roll coating methods because of low vapour pressures and correspondingly long drying times (>12 hours at room temperature).

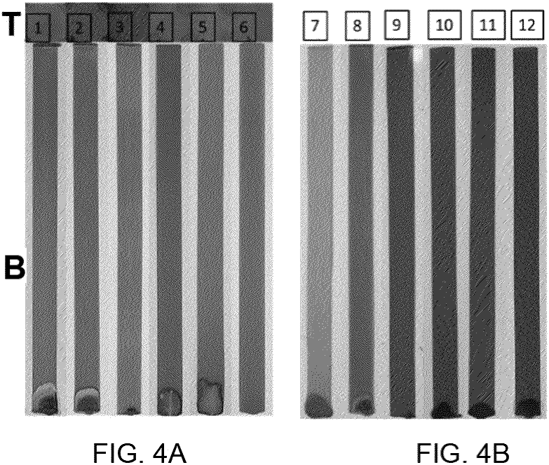

Evaluating solutions for scalable film deposition methods rather than spin-coating is important in demonstrating a material’s relevance for large scale manufacturing. Slot-die coating has been identified as a viable method for the roll-to-roll coating of organic semiconducting films with several reports detailing the practical use of the technique. [49-53] In all cases, using spin-coating or slot-die coating, the basic alcohol solutions of PDIN⁻ anion, uniform films were formed with no visible detects or particles (FIG. 4A). The purple solutions remained purple upon initial coating of the PET substrate, but as solvent evaporation occurred the resulting films were red in color.

Controlling film thickness is important in a variety of contexts, making it necessary to deposit films from a wide range of solution concentrations. Solutions of PDIN-H/NaOH were prepared with PDIN-H concentrations of 5, 10, 20, 30, 40, and 50 mg/mL with 1 molar equivalent of NaOH added. The solutions were purple in color with all solids dissolved. Upon slot-die coating, the inks yielded highly uniform thin films (FIG. 4B).

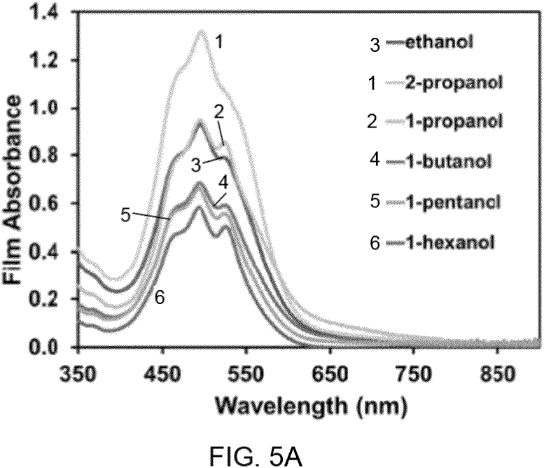

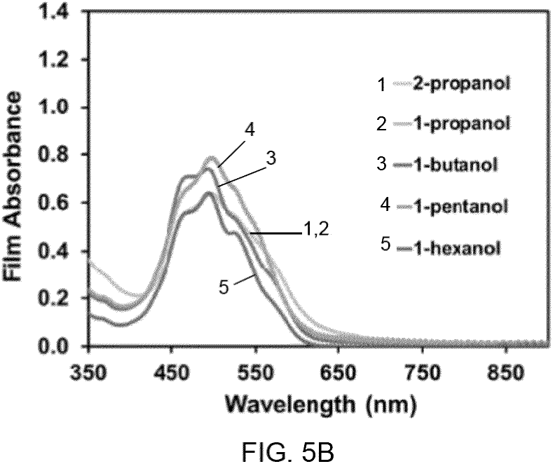

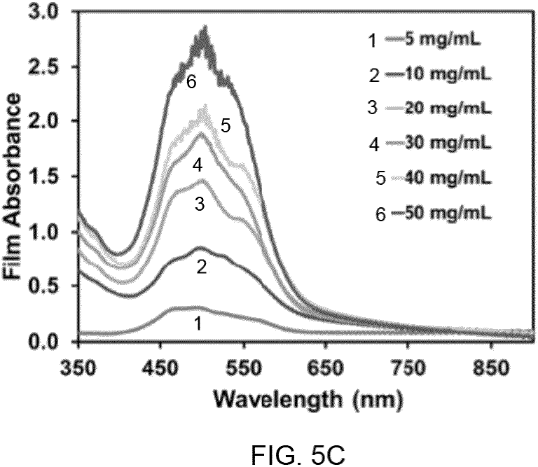

Optical absorption spectroscopy can be used to understand the molecular packing within the thin-film and determine relative film thickness based on light absorption, thus all films were analyzed using UV-visible spectroscopy (FIGS. 5A-C). All films exhibited the same basic optical absorption profile with a dominant absorption band from 380 nm to 625 nm with λₘₐₓ at 498 nm and a low energy shoulder at 524 nm. This profile has been seen previously in thin films of PDI monomers. [54, 55] For the spin-coated films an increase in light absorption is observed with an increase in the vapour pressure of the alcohol (FIG. 5A). This is consistent with quicker evaporation of higher vapor pressure solvents during spin-coating which leaves more material on the substrate. For the slot-die coated films from different alcohols, the optical absorption profiles remain constant, consistent with all material being deposited on the substrate (FIG. 5B). Increasing the concentration of PDIN-H from 5 to 50 mg/mL results in a progressive increase in light absorption (FIG. 5C). The optical absorption profile of an organic conjugated material in the film is known to be sensitive to morphology and aggregation. [56. 57] Because the optical profiles are more similar than different, major differences in the molecular packing are not expected.

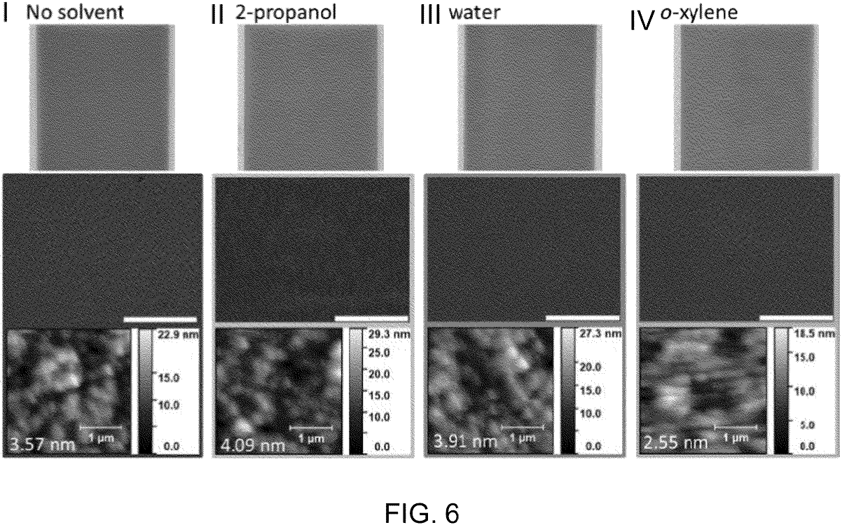

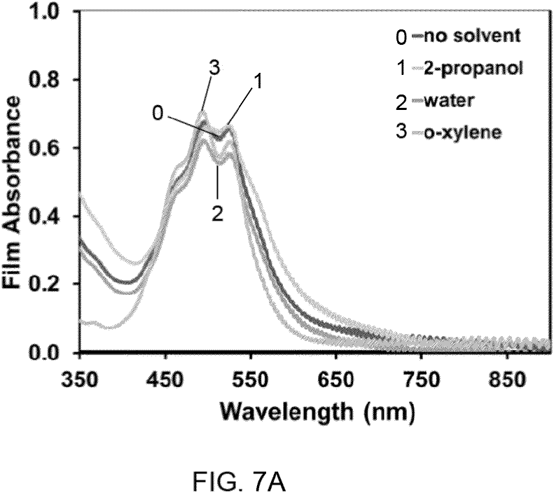

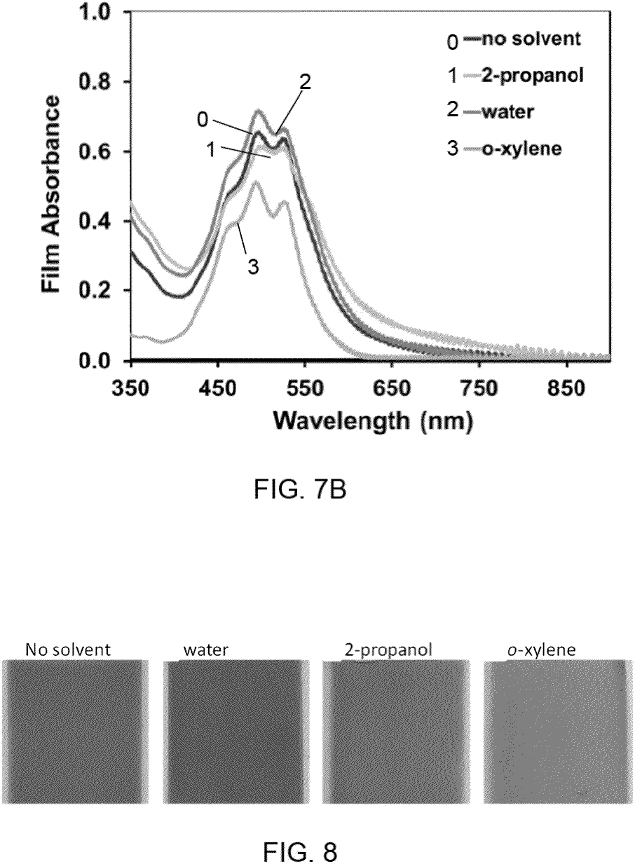

Multi-layers device formation requires that the deposited films are solvent resistant in respect to the subsequent layer. To test the films solvent resistance, the films were exposed to 2-propanol, water, and o-xylene. Films were inspected visually, examined using optical and atomic force microscopy, and UV-visible spectra were collected to assess film quality (FIGS. 6 and 7A and 7B). In all cases, no significant dissolution, swelling, cracking, or dewetting of the film was observed on exposure to solvent. Only with o-xylene did we observe any difference as seen in the slight change in the shape of the UV-visible optical absorption spectra. These results demonstrate that PDIN-H can be processed from greener solvents into solvent resistant films. These results were obtained whether the solvent treatment was applied via slot-die coating (FIGS. 6, 7A and 7B) or soaking the films in neat solvent for 10 seconds (FIG. 8).

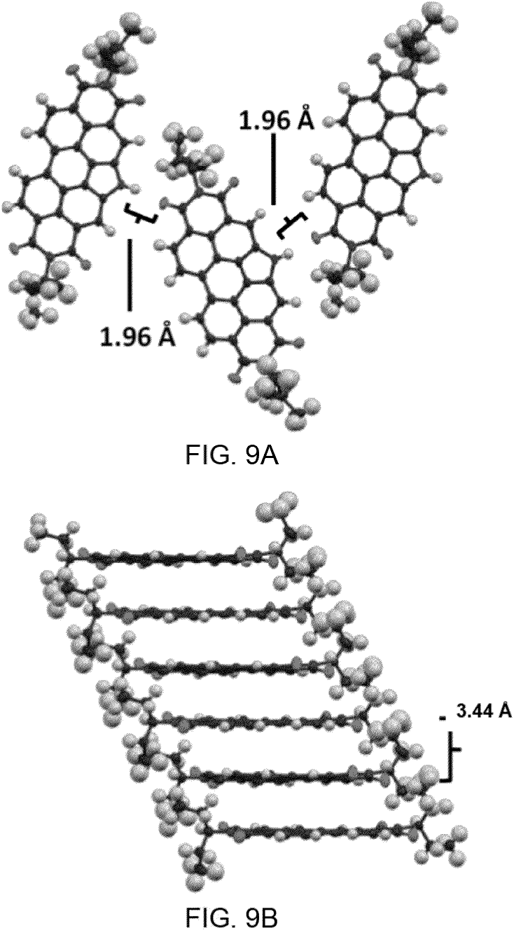

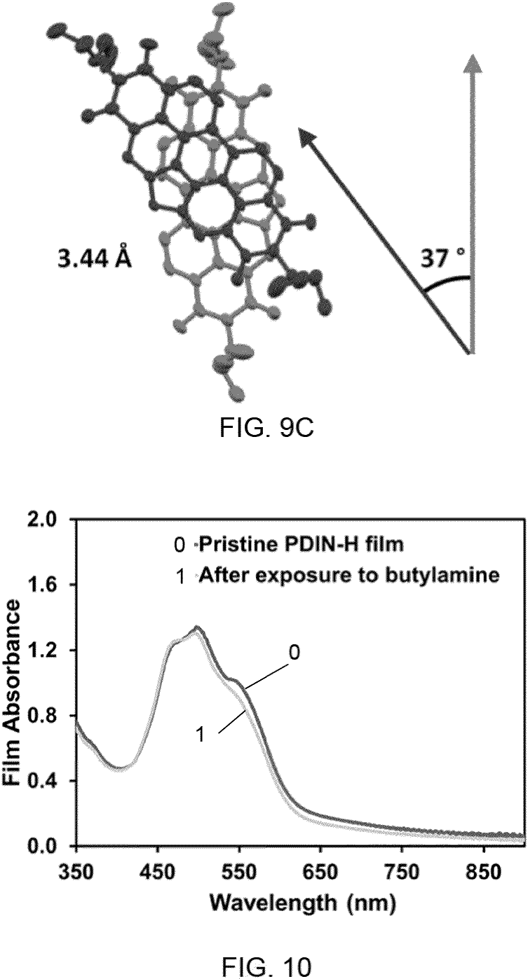

The solvent resistance of PDIN-H films likely results from hydrogen bonding between the NH and CO functional groups of adjacent molecules yielding strong intermolecular coupling. To assess this hypothesis, single crystals of PDIN-H were grown from dioxane/methanol solution (FIGS. 9A-C). The crystal structure of PDIN-H shows a distorted perylene core owing to the N-annulation bending the polycyclic aromatic structure. [22] Adjacent molecules of PDIN-H form nanoribbons tightly bound by hydrogen bonds with a NH··O distance of 1.96 Åand an angle of 171°. These parameters characterize the hydrogen bonds as moderate with a bond strength of ~ 4-15 kcal mol⁻¹. [58] In the π-stacking direction, PDIN-H forms slightly offset π-stacks with a π-π distance of 3.44 Å. The π-stacked molecules are twisted by 37° in addition to the offset to avoid clashing imide chains. Combined, the tight π-stacks, as well as the moderate strength hydrogen bonding, explain the solvent resistance of PDIN-H films. Further, the tight π-stacking indicates that PDIN-H can function as an electron transport material.

### Example 2: Preparation of Solvent Resistant Films Employing Primary Amine Base

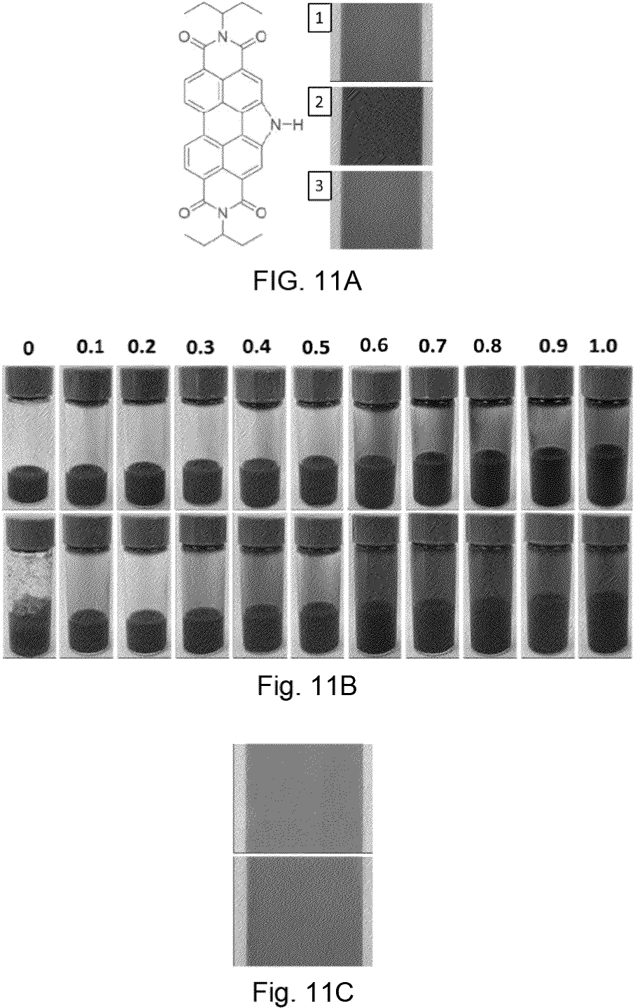

To replace hydroxide base in the processing of the PDIN-H films primary amines were tried. Primary amines are soluble in alcohols and water, and could interact with PDIN-H by polarizing the NH bond and thereby aid in both alcohol and water-based processing. [61] To test this, slot-die coated films of PDIN-H from 1-propanol/NaOH solution were exposed to butylamine vapor in a closed environment. Upon exposure to the butylamine vapor, the red PDIN-H film immediately changed color to purple. Simply removing the purple film from the amine vapor and letting the film sit in air for a few seconds re-established the red color (FIG. 11A). Here the excess butylamine deprotonates the PDIN-H to give the purple PDIN- anion. When the atmosphere saturated with butylamine is removed, the PDIN- anion is protonated regenerating the red color, characteristic of PDIN-H, while the butylamine evaporates. No changes in the film were detected by visual inspection and UV-visible spectroscopy (FIG. 10).

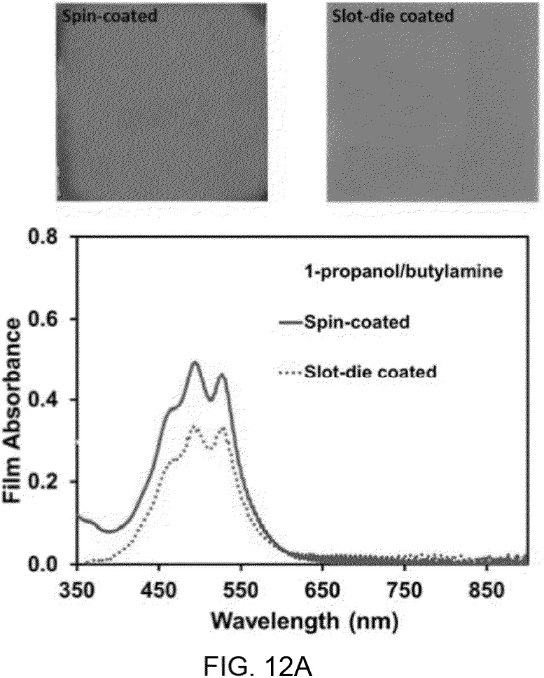

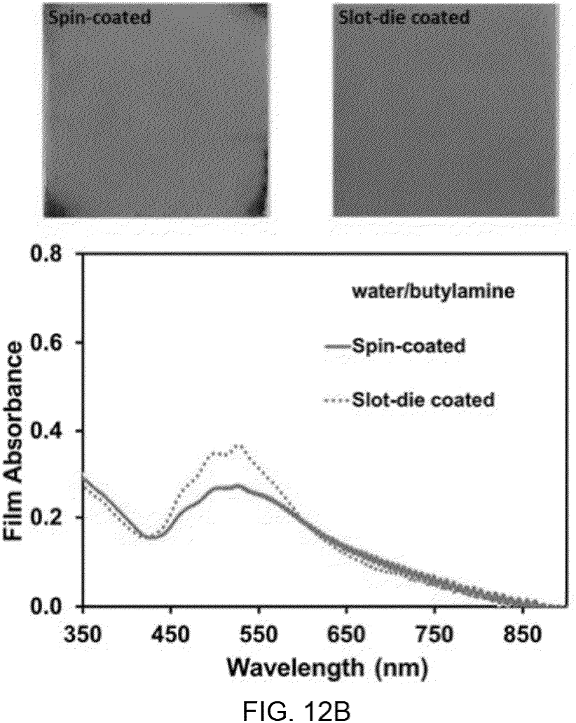

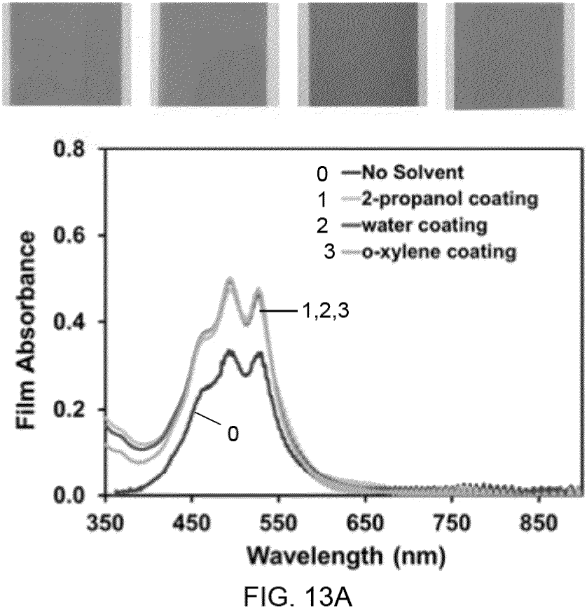

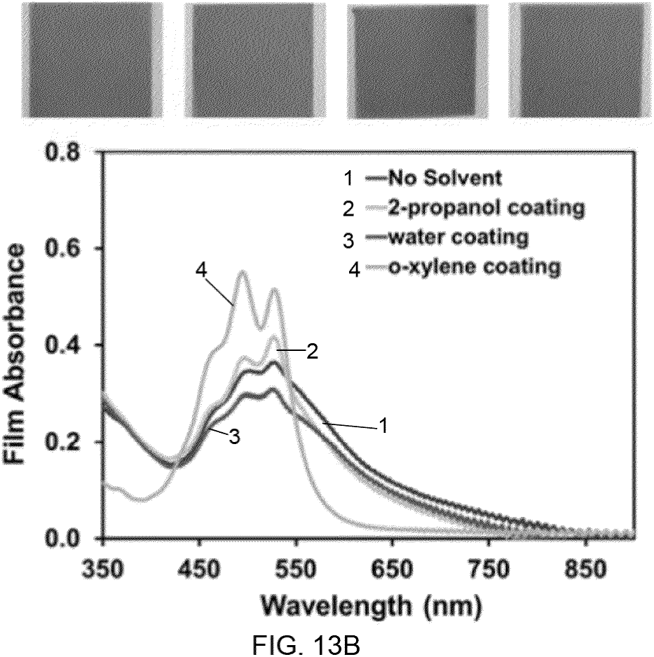

Next, slurries of PDIN-H in 1-propanol and water were prepared and volume equivalents of butylamine added. For both slurries there was a progressive color change and dissolution of the PDIN-H with increasing volume equivalents of butylamine added. At one volume equivalent complete dissolution was observed. (FIG. 11B). The solutions were readily processed into uniform thin-films via slot-die coating (FIG. 11C) or spin-coating (not shown). The films turned to the characteristic red color of PDIN-H immediately upon film drying. Analysis of the thin films via optical absorption spectroscopy showed the signature profile for the PDIN-H, although the water/butylamine processed films were slightly hazy as a result of light scattering (FIGS. 12A-B). PDIN-H can be readily processed into uniform thin-films using alcohol or water-based solutions with a volatile amine additive, thus eliminating the need for the use of caustic hydroxide salts. These films showed no significant signs of dissolution, swelling, or cracking upon coating polar and non-polar solvents on top (FIGS. 13A-13B).

### Example 3: Preparation of Solvent Resistant Films Employing Alkyl Ammonium Hydroxide Base

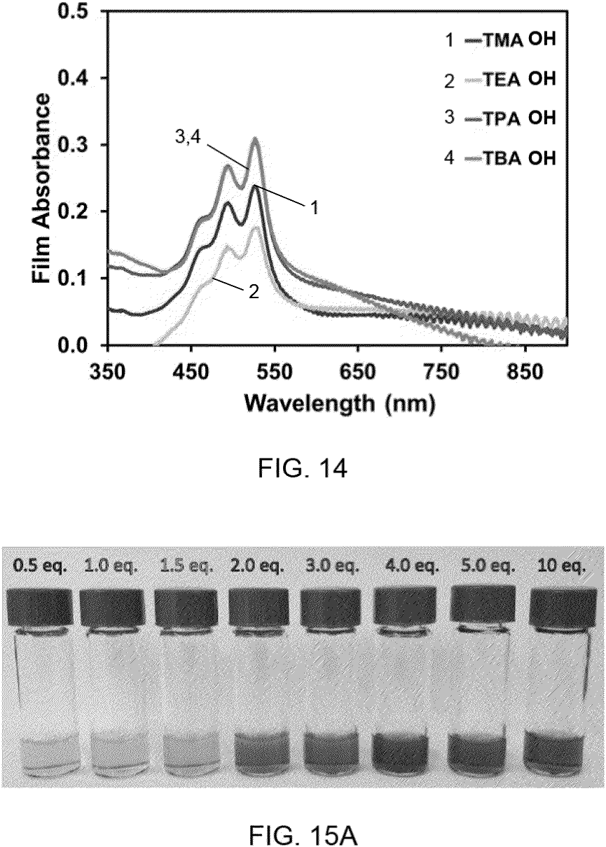

Films were prepared by slot-die roll coating from solutions containing 5 mg/mL of PDIN-H in 1-propanol with addition of 1 molar equivalent of a quaternary alkylammonium hydroxide base. The base was selected from tetramethylammonium hydroxide (TMAOH), tetraethylammonium hydroxide (TEAOH), tetra(n-propyl)ammonium hydroxide (TPAOH), or tetra(n-butyl)ammonium hydroxide (TBAOH). In all cases, PDIN-H dissolved in the alcohol on addition of the listed base and generally uniform films were prepared on processing. FIG. 14 provides a comparison of the UV-vis spectra of the films formed.

### Example 4: Preparation of Solvent Resistant Films Employing Alkali Metal Carbonates

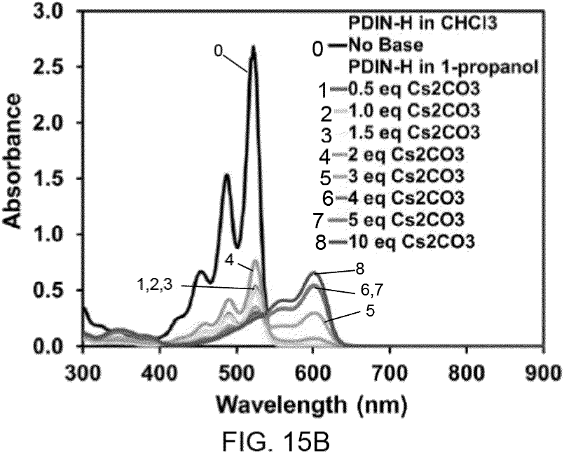

Films were prepared by slot-die coating from solutions containing 0.5 mg/mL of PDIN-H in 1-propanol with addition of varying amounts of CS₂CO₃. Films were slot-die coated in air at 170 µm/min pump rate and 330 mm/min chuck speed form solutions containing 0.5 to 10 eq. of the carbonate base. As in examples, addition of the carbonate base starting at 0.5 eq. resulted in a yellow solution which changed to reddish orange and ultimately to purple at 3.0 or more eq. (see FIG. 15A). FIG. 15B illustrates UV-vis spectra of the films prepared as a function of the equivalents of CS₂CO₃ added. The spectra in 1-propanol (1-8) are compared to the spectrum in CHCI₃ (0). As the number of equivalents of base increases the spectrum shifts toward purple (the anion). Based on the color of solutions prepared, films are preferably prepared from solutions containing about 2 or more equivalents of the carbonate base and more preferably between 2 and 4 equivalents of the carbonate base. The films prepared are found to be solvent resistant, particularly to water, 2-propanol and o-xylene.

### Example 5: Preparation of OPV Devices

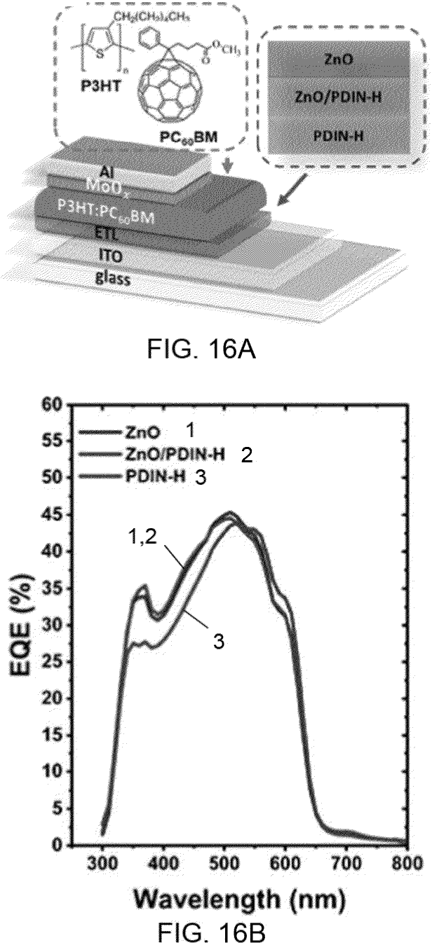

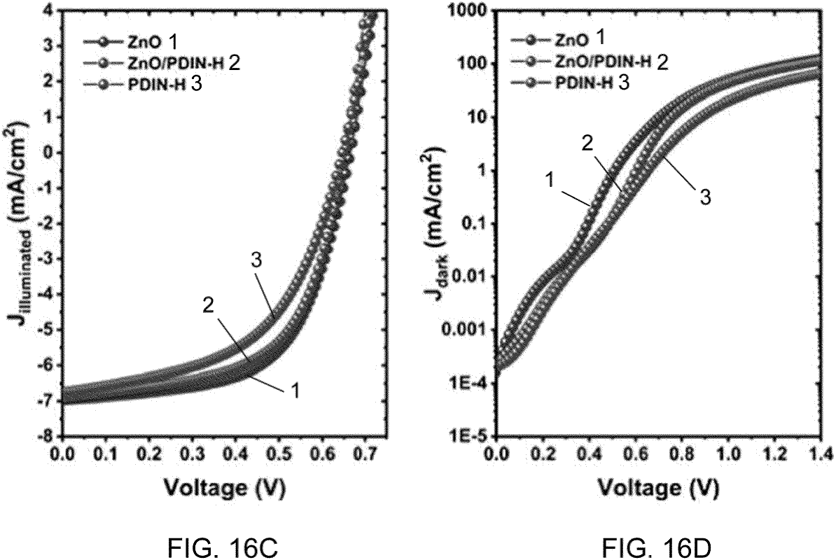

The semiconducting behavior of the PDIN-H films was assessed in organic photovoltaic (OPV) devices were fabricated. PDIN-H was used as an electron transport interlayer (ETL) in inverted type OPV devices to both modify and replace the standard ZnO ETL. A device architecture of glass/ITO/ETL/P3HT:PC60BM/MoOx/AI was utilized where the ETL was ZnO, ZnO/PDIN-H, or PDIN-H. PDIN-H was employed in the form of a film prepared as described herein. In all cases the PDIN-H film was formed by spin-coating a 5 mg/mL solution of PDIN-H in 1-propanol with 1 molar equivalent of NaOH added. The P3HT:PC60BM active layer was selected as it is common to nearly all OPV research laboratories. The device architecture is illustrated in FIG. 16A. The quantum efficiency (EQE) spectra, and current density-voltage plots are displayed in FIGS. 16B-16D and metrics are provided in the Table below. Control devices using a ZnO ETL gave a power conversion efficiency (PCE) of 2.8%, consistent with literature. Use of a ZnO/PDIN-H ETL gave similar PCE of 2.7%. The similar PCE is not unexpected as PDI based materials have been widely used to engineer the ZnO interface. [12, 67, 68] PDIN-H ETLs performed well, giving similar performance with OPV devices exhibiting a PCE of 2.3%. The slight drop owing to a slightly smaller fill factor (FF) (53% versus 60% in the control devices). A lower dark current at voltages > 0.6 V in case of the PDIN-H ETL in comparison to the ZnO and ZNO/PDIN-H ETLs indicates that the smaller FF is due to a higher series resistance at the ITO/PDIN-H interface. Regardless, the devices prepared demonstrate the utility of the alcohol solvent processed PDIN-H organic films to function as electron transport materials and provide a new material for all-organic PV device fabrication.

TABLE 1

OPV Device parameters (champion) Glass/ITO/ETL/P3HT:PC60BM/MoOx/AI

| ETL | J_{sc} (mA/cm2) | V_{oc} (mV) | FF [%] | PCE [%] |
|---|---|---|---|---|
| ZnO | 7.0 | 663 | 60 | 2.8 |
| ZnO/PDIN-H | 6.9 | 652 | 59 | 2.7 |
| PDIN-H | 6.8 | 646 | 53 | 2.3 |
| none | 8.3 | 577 | 41 | 1.9 |

### Organic Photovoltaics

Material Preparation: For ZnO sol-gel preparation, 197 mg zinc acetate was dissolved in a 6 mL ethanol plus 54 µL ethanolamine while mixing at 45° C. and 650 rpm for 50 min. To prepare the PDIN-Na solution, 10 mg/ml of sodium hydroxide (NaOH) was dissolved in 1-propanol through stirring at 400 rpm for 30 min. Then, 0.162 mL of this solution was added to a mixture of 1-propanol (2 mL) and PDIN-H (10 mg) followed by stirring at 400 rpm for 30 min. The solution was filtered through a 0.22 µm polypropylene filter prior to film formation. For the active layer, a 45 mg/ml solution of poly(3-hexylthiophene) (P3HT): [6,6]-Phenyl C61 butyric acid methyl ester (PC60BM), (1:1), in dichlorobenzene was prepared by stirring at 100° C. and 650 rpm overnight inside the glovebox. The solution was filtered through a 0.22 µm PTFE filter prior to film formation.

Device Fabrication: For OPV device fabrication, ITO-patterned glass substrates (Kintec) with a thickness of 80 nm and a sheet resistance of 15 Ω sq-1 were used. The substrates were first cleaned via sequential ultrasonic cleaning using Micro 90, deionized water, acetone and isopropanol solutions, following 5 min of O₂ plasma treatment. For coating the electron transport layers (ETL), ZnO was spin-coated at 1000 rpm for 60 s and annealed at 150° C. for 30 min, and PDIN-Na solution was spin coated at 4000 rpm for 60 s followed by annealing at 120° C. for 10 min. The P3HT:PC60BM solution was spin coated at 1000 rpm for 80 s and thermally annealed at 150° C. for 30 min inside the glovebox, resulting in a 150 nm thick bulk heterojunction (BHJ) active layer. The ITO-patterned substrate consisted of 12 OPVs with a surface area of 0.04 cm² of each. To compare the different ETLs more accurately, each substrate was first cut into four equal pieces, and the corresponding solution processes of each ETL were done on one piece. Then, the four pieces were taped together and transferred into an Angstrom Engineering EvoVac thermal evaporation chamber with a base pressure of 5 × 10-6 Torr for deposition of 5 nm of MoO₃ (American Elements), and 100 nm of aluminum anode (Angstrom Engineering).

Material and Device Characterization: Measurements of current density-voltage characteristics were carried out by a Keithley 2400 source, under 1-sun AM1.5G illumination from an ABET Sun 3000 Class AAA solar simulator. The external quantum efficiency (EQE) measurements were performed using the PV measurement (QEX10) system. The cells are illuminated under a monochromatic light, achieved though filtering a xenon arc lamp source, coupled with a germanium photodiode, by a dual-grating monochromator.

Additional description of device construction using film of this invention can be found in reference. 69 and the electronic supplementary information for that reference available from the publisher. Each of reference 69 and its supplementary information are incorporated by reference herein in its entirety for descriptions of OPV device structure and preparation.

Reference 70 provides a description of an OPV device configuration including a PDIN-H layer prepared as described herein. The reference describes an inverted organic solar cell where a PDIN-H film as described herein is inserted between the ZnO EEL and the organic active layer. BHJ active layer materials are P3HT and PC60BM. This reference and its supporting information available from the publisher are each incorporated by reference herein in its entirety for descriptions of device structure and construction.

### Example 6: General Coating Parameters

Spin-coated films were coated at room temperature at a speed of 1000 rpm for 60 s. Slot-die coated films were coated using a compact sheet coater from FOM Technologies equipped with a 13 mm wide slot-die head using a solution dispense rate ranging from 90 to 140 µL/min depending on solvent and a substrate motion speed of 30 cm/min.

Slot-die coating was performed with a compact sheet coater (FOM Technologies) under ambient conditions. For coatings the slot-die head was cleaned and assembled with 13 mm shim, then attached to the sheet coater. Approximately 2 mL of solution was loaded into a 5 mL Luer-Lok ™ (Becton, Dickinson, New Jersey) syringe and needle. The syringe was held upright and air expelled. The needle was removed, and the syringe was connected to P-628 Luer-Lok ™ female adapter then attached to 1521 FEP tubing (inner diameter: 1.75 mm) via a P-330X flangeless male nut and P-300X flangeless ferrule. Solution was pushed ¾ of way through tubing. The loaded syringe and tubing were inserted into a syringe holder on pump and the other end of tubing (with a P-330X flangeless male nut and P-300X flangeless ferrule) was attached to the slot die head for the final setup. Substrate, PET (Gloss waterproof inkjet film, Printing Supplies Direct) was loaded onto the flatbed and dust was removed via a contact cleaning hand roller (Teknek).

The slot-die head was moved to the starting point, lowered (until shim is around 1 cm from substrate) and the pump started (2 mL/min until solution was in the slot-die head. Printing conditions of pump dispense rate, length of print, and substrate motion speed were selected, pump dispense rate changing with various solvents, length of print changing according to length required, and substrate speed remaining constant at 30 cm/min. Once optimum dispense rate was selected (determined from previous testing), the pump was run until solution followed shim out of the slot-die head, pump was stopped, and meniscus was formed with substrate (by lowering slot-die head). Amount dispensed was zeroed on machine, pump was turned on followed immediately by the movement of the substrate.

After coating, the pump would be stopped about 1 s before the substrate finished moving to prevent significant pooling of the ink. The slot-die head was raised to break meniscus and moved accordingly to repeat process on another part of substrate, if required. All substrates were allowed at least 30 minutes to dry on the flatbed before removal, cut to 1.5 cm × 1.5 cm samples after an hour, and allowed to dry in petri dishes overnight in a drawer before being subject to analysis. For slot-die coated solvent resistant tests neat solvents were loaded into a cleaned syringe and coated directly on top of previously coated films.

### Example 7: Materials and Methods

Additional details of the preparation and testing of films of this invention can be found in reference 69 and the electronic supplementary information for that reference available from the publisher.

### UV-Visible Spectroscopy (UV-Vis)

Measurements were recorded using an Agilent Technologies Cary 60 UV-Vis spectrometer at room temperature. All solution UV-Vis experiments were run using 10 mm quartz cuvettes. Films were spin-coated onto PET substrates.

### Single Crystal X-Ray Crystallography

Single crystals of C34H29N3O4 PDIN-H were grown by vapor diffusion at room temperature of methanol into a solution of PDIN-H in dioxane. A suitable crystal was selected and mounted on a glass loop using Paratone oil. Diffraction experiments were performed on a Bruker Smart diffractometer equipped with an Incoatec Microfocus (Cu Ka, A = 1.54178 A) and an APEX II CCD detector. The crystal was kept at 173 K during data collection. Diffractions spots were integrated and scaled with SAINT [62] and the space group was determined with XPREP [63]. Using Olex2 [64], the structure was solved with the ShelXT [65] structure solution program using Intrinsic Phasing and refined with the ShelXL [66] refinement package using Least Squares minimization.

Crystal Data for C₃₄H₂₉N₃O₄ (M =543.60 g/mol): monoclinic, space group P21 /c (no. 14), a = 18.2166(7) A, b = 18.5973(10) Å, c = 7.8095(2) A, 13 = 97.881(2)°, V = 2620.71(19) A3, Z = 4, T = 173 K, u(CuKa) = 0.734 mm-1, Dcalc = 1.378 g/cm3, 22039 reflections measured (4.898° <_ 20 <_ 130.452°), 4467 unique (Rint = 0.0349, Rsigma = 0.0216) which were used in all calculations. The final R1 was 0.0581 (I > 2_{Q}(I)) and wR2 was 0.1639 (all data).

### Atomic Force Microscopy (AFM)

AFM measurements were performed by using a TT2- AFM (AFM Workshop) in tapping mode and WSxM software with a tip at a resonance frequency of 300 kHz, a force constant of 40 N/m and a reflective back side aluminum coating (Tap300AI-G, BudgetSensors).

### Optical Light Microscopy

Images were taken using a BX53 Olympus Scope.

### References

(1) Gsänger, M.; Bialas, D.; Huang, L.; Stolte, M.; Wurthner, F. Organic Semiconductors Based on Dyes and Color Pigments. Adv. Mater. 2016, 28 (19), 3615-3645. https://doi.org/10.1002/adma.201505440.
(2) Greene, M. Perylene Pigments. In High Performance Pigments; John Wiley & Sons, Ltd, 2003; pp 249-261. https://doi.org/10.1002/3527600493.ch16.
(3) Dayneko, S. V.; Pahlevani, M.; Welch, G. C. Indoor Photovoltaics: Photoactive Material Selection, Greener Ink Formulations, and Slot-Die Coated Active Layers. ACS Appl. Mater. Interfaces 2019, 11 (49), 46017-46025. https://doi.org/10.1021/acsami.9b19549.
(4) Zhou, Y.; Xue, B.; Wu, C.; Chen, S.; Liu, H.; Jiu, T.; Li, Z.; Zhao, Y. Sulfur-Substituted Perylene Diimides: Efficient Tuning of LUMO Levels and Visible-Light Absorption via Sulfur Redox. Chem. Commun. 2019, 55 (90), 13570-13573. https://doi.org/10.1039/C9CC07040K.
(5) Rocard, L.; Goujon, A.; Hudhomme, P. Nitro-Perylenediimide: An Emerging Building Block for the Synthesis of Functional Organic Materials. Molecules 2020, 25 (6), 1402. https://doi.org/10.3390/molecules25061402.
(6) Percec, V.; Peterca, M.; Tadjiev, T.; Zeng, X.; Ungar, G.; Leowanawat, P.; Aqad, E.; Imam, M. R.; Rosen, B. M.; Akbey, U.; Graf, R.; Sekharan, S.; Sebastiani, D.; Spiess, H. W.; Heiney, P. A.; Hudson, S. D. Self-Assembly of Dendronized Perylene Bisimides into Complex Helical Columns. J. Am. Chem. Soc. 2011, 133 (31), 12197-12219. https://doi.org/10.1021/ja204366b.
(7) Schwartz, P.-O.; Biniek, L.; Zaborova, E.; Heinrich, B.; Brinkmann, M.; Leclerc, N.; Mery, S. Perylenediimide-Based Donor-Acceptor Dyads and Triads: Impact of Molecular Architecture on Self-Assembling Properties. J. Am. Chem. Soc. 2014, 136 (16), 5981-5992. https://doi.org/10.1021/ja4129108.
(8) Zhang, J.; Bai, F.; Li, Y.; Hu, H.; Liu, B.; Zou, X.; Yu, H.; Huang, J.; Pan, D.; Ade, H.; Yan, H. Intramolecular rr-Stacked Perylene-Diimide Acceptors for Non-Fullerene Organic Solar Cells. J. Mater. Chem. A 2019, 7 (14), 8136-8143. https://doi.org/10.1039/C9TA00343 F.
(9) Nowak-Krol, A.; Shoyama, K.; Stolte, M.; Wurthner, F. Naphthalene and Perylene Diimides - Better Alternatives to Fullerenes for Organic Electronics? Chem. Commun. 2018, 54 (98), 13763-13772. https://doi.org/10.1039/C8CC07640E.
(10) Kozma, E.; Mroz, W.; Villafiorita-Monteleone, F.; Galeotti, F.; Andicsova-Eckstein, A.; Catellani, M.; Botta, C. Perylene Diimide Derivatives as Red and Deep Red-Emitters for Fully Solution Processable OLEDs. RSC Adv. 2016, 6 (66), 61175-61179. https://doi.org/10.1039/C6RA10467C.
(11) Weitz, R. T.; Amsharov, K.; Zschieschang, U.; Villas, E. B.; Goswami, D. K.; Burghard, M.; Dosch, H.; Jansen, M.; Kern, K.; Klauk, H. Organic N-Channel Transistors Based on Core-Cyanated Perylene Carboxylic Diimide Derivatives. J. Am. Chem. Soc. 2008, 130 (14), 4637-4645. https://doi.org/10.1021/ja074675e.
(12) Wurthner, F.; Saha-Moller, C. R.; Fimmel, B.; Ogi, S.; Leowanawat, P.; Schmidt, D. Perylene Bisimide Dye Assemblies as Archetype Functional Supramolecular Materials. Chem. Rev. 2016, 116 (3), 962-1052. https://doi.org/10.1021 /acs.chemrev.5b00188.
(13) Chen, S.; Slattum, P.; Wang, C.; Zang, L. Self-Assembly of Perylene Imide Molecules into 1D Nanostructures: Methods, Morphologies, and Applications. Chem. Rev. 2015, 115 (21), 11967-11998. https://doi.org/10.1021/acs.chemrev.5b00312.
(14) Li, G.; Li, D.; Liu, X.; Xu, H.; Zhang, J.; Wang, S.; Liu, Z.; Tang, B. Novel Dithiano-Thieno Fused Perylene Diimides: Synthesis, Characterization and Application in Organic Thin-Film Transistors (OTFTs). Chem. Commun. 2019, 55 (65), 9661-9664. https://doi.org/10.1039/C9CC04133H.
(15) Luo, Z.; Liu, T.; Cheng, W.; Wu, K.; Xie, D.; Huo, L.; Sun, Y.; Yang, C. A Three-Dimensional Thiophene-Annulated Perylene Bisimide as a Fullerene-Free Acceptor for a High Performance Polymer Solar Cell with the Highest PCE of 8.28% and a VOC over 1.0 V. J. Mater. Chem. C 2018, 6 (5), 1136-1142. https://doi.org/10.1039/C7TC05261H.
(16) Meng, D.; Sun, D.; Zhong, C.; Liu, T.; Fan, B.; Huo, L.; Li, Y.; Jiang, W.; Choi, H.; Kim, T.; Kim, J. Y.; Sun, Y.; Wang, Z.; Heeger, A. J. High-Performance Solution-Processed Non-Fullerene Organic Solar Cells Based on Selenophene-Containing Perylene Bisimide Acceptor. J. Am. Chem. Soc. 2016, 138 (1), 375-380. https://doi.org/10.1021/jacs.5b11149.
(17) Yin, Y.; Song, J.; Guo, F.; Sun, Y.; Zhao, L.; Zhang, Y. Asymmetrical vs Symmetrical Selenophene-Annulated Fused Perylenediimide Acceptors for Efficient Non-Fullerene Polymer Solar Cells. ACS Appl. Energy Mater. 2018, 1 (11), 6577-6585. https://doi.org/10.1021/acsaem.8b01484.
(18) You, F.; Zhou, X.; Huang, H.; Liu, Y.; Liu, S.; Shao, J.; Zhao, B.; Qin, T.; Huang, W. N -Annulated Perylene Diimide Derivatives as Non-Fullerene Acceptors for Solution-Processed Solar Cells with an Open-Circuit Voltage of up to 1.14 V. New J. Chem. 2018, 42 (18), 15079-15087. https://doi.org/10.1039/C8NJ02566E.
(19) Langhals, H.; Kirner, S. Novel Fluorescent Dyes by the Extension of the Core of Perylenetetracarboxylic Bisimides. Eur. J. Org. Chem. 2000, 2000 (2), 365-380. https://doi . org/10.1002/(S I C I ) 1099-0690(200001 )2000:2 <365::AI D-EJOC365>3.0.CO;2-R.
(20) Gupta, R. K.; Dey, A.; Singh, A.; Iyer, P. K.; Sudhakar, A. A. Heteroatom Bay-Annulated Perylene Bisimides: New Materials for Organic Field Effect Transistors. ACS Appl. Electron. Mater. 2019, 1 (8), 1378-1386. https://doi.org/10.1021/acsaelm.9b00004.
(21) Gupta, R. K.; Shankar Rao, D. S.; Prasad, S. K.; Achalkumar, A. S. Columnar Self-Assembly of Electron-Deficient Dendronized Bay-Annulated Perylene Bisimides. Chem. - Eur. J. 2018, 24 (14), 3566-3575. https://doi.org/10.1002/chem.201705290.
(22) Hendsbee, A. D.; Sun, J.-P.; Law, W. K.; Yan, H.; Hill, !. G.; Spasyuk, D. M.; Welch, G. C. Synthesis, Self-Assembly, and Solar Cell Performance of N-Annulated Perylene Diimide Non-Fullerene Acceptors. Chem. Mater. 2016, 28 (19), 7098-7109. https://doi.org/10.1021/acs.chemmater.6b03292.
(23) McAfee, S. M.; Dayneko, S. V.; Josse, P.; Blanchard, P.; Cabanetos, C.; Welch, G. C. Simply Complex: The Efficient Synthesis of an Intricate Molecular Acceptor for High-Performance Air-Processed and Air-Tested Fullerene-Free Organic Solar Cells. Chem. Mater. 2017, 29 (3), 1309-1314. https://doi.org/10.1021/acs.chemmater.6b04862.
(24) Dayneko, S. V.; Hendsbee, A. D.; Welch, G. C. Fullerene-Free Polymer Solar Cells Processed from Non-Halogenated Solvents in Air with PCE of 4.8%. Chem. Commun. 2017, 53 (6), 1164-1167. https://doi.org/10.1039/C6CC08939A.
(25) Payne, A.-J.; Li, S.; Dayneko, S.V; Risko, C.; Welch, G.C. An Unsymmetrical Non-Fullerene Acceptor: Synthesis via Direct Heteroarylation, Self-Assembly, and Utility as a Low Energy Absorber in Organic Photovoltaic Cells. Chem. Commun. Camb. Engl. 2017, 53 (73), 10168-10171. https://doi.org/10.1039/c7cc05836e.
(26) McAfee, S. M.; Payne, A.-J.; Dayneko, S. V.; Kini, G. P.; Song, C. E.; Lee, J.-C.; Welch, G. C. A Non-Fullerene Acceptor with a Diagnostic Morphological Handle for Streamlined Screening of Donor Materials in Organic Solar Cells. J. Mater. Chem. A 2017, 5 (32), 16907-16913. https://doi.org/10.1039/C7TA05282K.
(27) McAfee, S. M.; Dayneko, S. V.; Hendsbee, A. D.; Josse, P.; Blanchard, P.; Cabanetos, C.; Welch, G. C. Applying Direct Heteroarylation Synthesis to Evaluate Organic Dyes as the Core Component in PDI-Based Molecular Materials for Fullerene-Free Organic Solar Cells. J. Mater. Chem. A 2017, 5 (23), 11623-11633. https://doi.org/10.1039/C7TA00318H.
(28) Dayneko, S. V.; Hendsbee, A. D.; Welch, G. C. Combining Facile Synthetic Methods with Greener Processing for Efficient Polymer-Perylene Diimide Based Organic Solar Cells. Small Methods 2018, 2 (6), 1800081. https://doi.org/10.1002/smtd.201800081.
(29) McAfee, S. M.; Payne, A.-J.; Hendsbee, A. D.; Xu, S.; Zou, Y.; Welch, G. C. Toward a Universally Compatible Non-Fullerene Acceptor: Multi-Gram Synthesis, Solvent Vapor Annealing Optimization, and BDT-Based Polymer Screening. Sol. RRL 2018, 2 (9), 1800143. https://doi.org/10.1002/solr.201800143.
(30) McAfee, S. M.; Welch, G. C. Development of Organic Dye-Based Molecular Materials for Use in Fullerene-Free Organic Solar Cells. Chem. Rec. 2019, 19 (6), 989-1007. https://doi.org/10.1002/tcr.201800114.
(31) Tintori, F.; Laventure, A.; Welch, G. C. Perylene Diimide Based Organic Photovoltaics with Slot-Die Coated Active Layers from Halogen-Free Solvents in Air at Room Temperature. ACS Appl. Mater. Interfaces 2019, 11 (42), 39010-39017. https://doi.org/10.1021/acsami.9b14251.
(32) Dayneko, S. V.; Pahlevani, M.; Welch, G. C. Indoor Photovoltaics: Photoactive Material Selection, Greener Ink Formulations, and Slot-Die Coated Active Layers. ACS Appl. Mater. Interfaces 2019, 11 (49), 46017-46025. https://doi.org/10.1021/acsami.9b19549.
(33) Irimia-Vladu, M.; Glowacki, Eric. D.; Voss, G.; Bauer, S.; Sariciftci, N. S. Green and Biodegradable Electronics. Mater. Today 2012, 15 (7), 340-346. https://doi.org/10.1016/S1369-7021(12)70139-6.
(34) Irimia-Vladu, M. “Green” Electronics: Biodegradable and Biocompatible Materials and Devices for Sustainable Future. Chem. Soc. Rev. 2014, 43 (2), 588-610. https://doi.org/10.1039/C3CS60235D.
(35) Burke, D. J.; Lipomi, D. J. Green Chemistry for Organic Solar Cells. Energy Environ. Sci. 2013, 6 (7), 2053-2066. https://doi.org/10.1039/C3EE41096J.
(36) Po, R.; Roncali, J. Beyond Efficiency: Scalability of Molecular Donor Materials for Organic Photovoltaics. J. Mater. Chem. C 2016, 4 (17), 3677-3685. https://doi.org/10.1039/C5TC03740A.
(37) Diao, Y.; Shaw, L.; Bao, Z.; Mannsfeld, S. C. B. Morphology Control Strategies for Solution-Processed Organic Semiconductor Thin Films. Energy Environ. Sci. 2014, 7 (7), 2145-2159. https://doi.org/10.1039/C4EE00688G.
(38) Ma, Z.; Zhao, B.; Gong, Y.; Deng, J.; Tan, Z. Green-Solvent-Processable Strategies for Achieving Large-Scale Manufacture of Organic Photovoltaics. J. Mater. Chem. A 2019, 7 (40), 22826-22847. https://doi.org/10.1039/C9TA09277C.
(39) Zhang, S.; Ye, L.; Zhang, H.; Hou, J. Green-Solvent-Processable Organic Solar Cells. Mater. Today 2016, 19 (9), 533-543. https://doi.org/10.1016/j.mattod.2016.02.019.
(40) Schmatz, B.; Yuan, Z.; Lang, A. W.; Hernandez, J. L.; Reichmanis, E.; Reynolds, J. R. Aqueous Processing for Printed Organic Electronics: Conjugated Polymers with Multistage Cleavable Side Chains. ACS Cent. Sci. 2017, 3 (9), 961-967. https://doi.org/10.1021/acscentsci.7b00232.
(41) Khau, B. V.; Savagian, L. R.; De Keersmaecker, M.; Gonzalez, M. A.; Reichmanis, E. Carboxylic Acid Functionalization Yields Solvent-Resistant Organic Electrochemical Transistors. ACS Mater. Lett. 2019, 1 (6), 599-605. https://doi.org/10.1021/acsmaterialslett.9b00373.
(42) Li, Z.; Ying, L.; Zhu, P.; Zhong, W.; Li, N.; Liu, F.; Huang, F.; Cao, Y. A Generic Green Solvent Concept Boosting the Power Conversion Efficiency of All-Polymer Solar Cells to 11%. Energy Environ. Sci. 2019, 12 (1), 157-163. https://doi.org/10.1039/C8EE02863J.
(43) Zhang, C.; Holdcroft, S. Photocrosslinking of Low Band-Gap Conjugated Polymers Using Alkyl Chloride Sidechains: Toward High-Efficiency, Thermally Stable Polymer Solar Cells. J. Mater. Res. 2018, 33 (13), 1879-1890. https://doi.org/10.1557/jmr.2018. 78.
(44) Png, R.Q.; Chia, P.J.; Tang, J.C.; Liu, B.; Sivaramakrishnan, S.; Zhou, M.; Khong, S.H.; Chan, H.S.; Burroughes, J.H.; Chua, L.L; Friend, R.H.; Ho, P.K. High-performance polymer semiconducting heterostructure devices by nitrene-mediated photocrosslinking of alkyl side chains. Nat. Mater. 2010, 9(2), 152-158. https://doi.org/10.1038/nmat2594.
(45) Rumer, J.W.; McCulloch, !. Organic photovoltaics: Crosslinking for optimal morphology and stability. Mater. Today. 2015, 18 (8), 425-435. https://doi.org/10.1016/j. mattod.2015.04.001
(46) Hunger, K. Industrial Dyes, 1st Ed; Wiley -VCH: Weinheim, Germany, 2003.
(47) Wen, X.; Nowak-Krol, A.; Nagler, O.; Kraus, F.; Zhu, N.; Zheng, N.; Muller, M.; Schmidt, D.; Xie, Z.; Wurthner, F. Tetrahydroxy-Perylene Bisimide Embedded in a Zinc Oxide Thin Film as an Electron-Transporting Layer for High-Performance Non-Fullerene Organic Solar Cells. Angew. Chem. Int. Ed. 2019, 58 (37), 13051-13055. https://doi.org/10.1002/anie.201907467.
(48) He, E.; Wang, J.; Xu, H.; He, Z.; Wang, H.; Zhao, H.; Zhang, Y.; Zhang, R.; Zhang, H. Facile Synthesis of Graphene Oxide Sheet-Immobilized Perylene Diimide Radical Anion Salt and Its Optical Response to Different Solvents and PH Values. J. Mater. Sci. 2016, 51 (14), 6583-6589. https://doi.org/10.1007/s10853-016-9885-8.
(49) Sondergaard, R.; Hosel, M.; Angmo, D.; Larsen-Olsen, T. T.; Krebs, F. C. Roll-to-Roll Fabrication of Polymer Solar Cells. Mater. Today 2012, 15 (1), 36-49. https://doi.org/10.1016/S1369-7021 (12)70019-6.
(50) Sandstrom, A.; Dam, H. F.; Krebs, F. C.; Edman, L. Ambient Fabrication of Flexible and Large-Area Organic Light-Emitting Devices Using Slot-Die Coating. Nat. Commun. 2012, 3 (1), 1-5. https://doi.org/10.1038/ncomms2002.
(51) Alstrup, J.; Jorgensen, M.; Medford, A. J.; Krebs, F. C. Ultra Fast and Parsimonious Materials Screening for Polymer Solar Cells Using Differentially Pumped Slot-Die Coating. ACS Appl. Mater. Interfaces 2010, 2 (10), 2819-2827. https://doi.org/10.1021 /am100505e.
(52) Krebs, F. C. Polymer Solar Cell Modules Prepared Using Roll-to-Roll Methods: Knife-over-Edge Coating, Slot-Die Coating and Screen Printing. Sol. Energy Mater. Sol. Cells 2009, 93 (4), 465-475. https://doi.org/10.1016/j.solmat.2008.12.012.
(53) Hosel, M.; Dam, H. F.; Krebs, F. C. Development of Lab-to-Fab Production Equipment Across Several Length Scales for Printed Energy Technologies, Including Solar Cells. Energy Technol. 2015, 3 (4), 293-304. https://doi.org/10.1002/ente.201402140.
(54) Namepetra, A.; Kitching, E.; Eftaiha, A. F.; Hill, I. G.; Welch, G. C. Understanding the Morphology of Solution Processed Fullerene-Free Small Molecule Bulk Heterojunction Blends. Phys. Chem. Chem. Phys. 2016, 18 (18), 12476-12485. https://doi.org/10.1039/C6CP01269H.
(55) Nazari, M.; Cieplechowicz, E.; Welsh, T. A.; Welch, G. C. A Direct Comparison of Monomeric vs. Dimeric and Non-Annulated vs. N-Annulated Perylene Diimide Electron Acceptors for Organic Photovoltaics. New J. Chem. 2019, 43 (13), 5187-5195. https://doi.org/10.1039/C8NJ06491A.
(56) Spano, F. C. The Spectral Signatures of Frenkel Polarons in H- and J-Aggregates. Acc. Chem. Res. 2010, 43 (3), 429-439. https://doi.org/10.1021/ar900233v.
(57) Bencheikh, F.; Duche, D.; Ruiz, C. M.; Simon, J.-J.; Escoubas, L. Study of Optical Properties and Molecular Aggregation of Conjugated Low Band Gap Copolymers: PTB7 and PTB7-Th. J. Phys. Chem. C 2015, 119 (43), 24643-24648. https://doi.org/10.1021/acs.jpcc.5b07803.
(58) Steiner, T. W. The Hydrogen Bond in the Solid State. Angew. Chem. 2002. https://doi.org/10.1002/1521-3773(20020104)41:1 <48::AID-ANIE48>3.0.CO;2-U.
(59) Xie, Z.; Wurthner, F. Hybrid Photoconductive Cathode Interlayer Materials Composed of Perylene Bisimide Photosensitizers and Zinc Oxide for High Performance Polymer Solar Cells. Adv. Energy Mater. 2017, 7 (16), 1602573. https://doi.org/10.1002/aenm.201602573.
(60) Abd-Ellah, M.; Cann, J.; Dayneko, S. V.; Laventure, A.; Cieplechowicz, E.; Welch, G. C. Interfacial ZnO Modification Using a Carboxylic Acid Functionalized N-Annulated Perylene Diimide for Inverted Type Organic Photovoltaics. ACS Appl. Electron. Mater. 2019, 1 (8), 1590-1596. https://doi.org/10.1021/acsaelm.9b00328.
(61) Vespa, M.; Cann, J. R.; Dayneko, S. V.; Melville, O. A.; Hendsbee, A. D.; Zou, Y.; Lessard, B. H.; Welch, G. C. Synthesis of a Perylene Diimide Dimer with Pyrrolic N-H Bonds and N-Functionalized Derivatives for Organic Field-Effect Transistors and Organic Solar Cells. Eur. J. Org. Chem. 2018 (33), 4592-4599. https://doi.org/10.1002/ejoc.201801055.
(62) Bruker-AXS. SAINT; Madison, Wisconsin, USA, 2017.
(63) Bruker-AXS. XPREP; Madison, Wisconsin, USA, 2017.
(64) Dolomanov, O.V.; Bourhis, L.J.; Gildea, R.J.; Howard, J.A.K.; Puschmann, H.. OLEX2: A Complete Structure Solution, Refinement and Analysis Program. J. Appl. Cryst. 2009, 42, 339-341.
(65) Sheldrick, G.M., SHELXT-Integrated space-group and crystal-structure determination, Acta Cryst. 2015, A71, 3-8.
(66) Sheldrick, G.M, Crystal structure refinement with SHELXL. Acta Cryst. 2015, C71, 3-8.
(67) Z. Xie and F. Wurthner, Hybrid Photoconductive Cathode Interlayer Materials Composed of Perylene Bisimide Photosensitizers and Zinc Oxide for High Performance Polymer Solar Cells, Adv. Energy Mater., 2017, 7(16), 1602573.
(68) M. Abd-Ellah, J. Cann, S. V. Dayneko, A. Laventure, E. Cieplechowicz and G. C. Welch, Interfacial ZnO Modification Using a Carboxylic Acid Functionalized N-Annulated Perylene Diimide for Inverted Type Organic Photovoltaics, ACS Appl. Electron. Mater., 2019, 1(8), 1590-1596.
(69) Harding, C.R., Cann, J., Laventure, A., Sadeghianlemraski, M, Abd-Ellah, M., Rao, K.R., Gelfand, B.S., Aziz, H., Kaake, L., Risko C., Gregory C. Welch G.C., Acid dyeing for green solvent processing of solvent resistant semiconducting organic thin films, Materials Horizons, 2020, 7:2959-2969 and electronic supplementary information for this reference available from the publisher, see DOI: 10.1039/d0mh00785d.
(70) Sadeghianlemraski, M, Harding, C.R., Welch G. C., Aziz, H. Significant Photostability Enhancement of Inverted Organic Solar Cells by Inserting an N-Annulated Perylene Diimide (PDIN-H) between the ZnO Electron Extraction Layer and the Organic Active Layer, ACS Applied Energy Materials, 2020, 3:11655-11665.
(71) Lai, T-H, Tsang, S-W., Manders, J.R., Chen, S., So, F., Properties of Interface Layers of Organic Photovoltaics, 2013, Materials Today 16(11): 424-432.

1. A method for preparing a film comprising an N-annulated perylene diimide (PDI) compound having a pyrrole N—H bond comprising:
dissolving a selected amount of the N-annulated PDI compound in a selected amount of a solvent selected from water, a C₁-C₆ alcohol or a miscible combination thereof by addition to the solvent containing the N-annulated compound of an amount of base at least sufficient to polarize the pyrrole N—H bond giving an ionic salt dissolved in the solvent to form a film-precursor solution;
forming a layer using the film-precursor solution; and
removing solvent from the layer to form the film.

2. The method of claim 1, wherein the N-annulated PDI compound is a compound of Formula I: 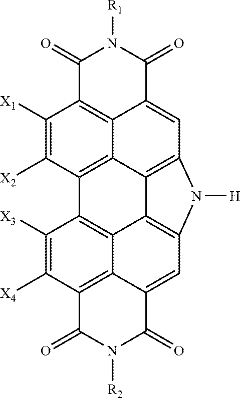
wherein:
R₁ and R₂ are independently a substituted or unsubstituted C₁ to C₁₈ linear or branched alkyl; and
X₁-X₄ are independently selected from H, a C₁-C₆ substituted or unsubstituted alkyl, a halogen, NO₂, or CN or X₂ and X₃ together form —S—S— and X₁ and X₄ are independently selected from H, a C₁-C₆ substituted or unsubstituted alkyl, a halogen, NO₂, or CN;
wherein optional substitution of alkyl groups is substitution with one or more halogens, —CN, —NO₂, —C(O)R′, —COOR′, —C(O)NH₂, —NHC(O)R′, —C(O)NR′R″, —CF₃, —SO₃H, —SO₂CF3, —SO₂R′, —SO₂NR′R′’, —OR′, —OC(O)R′, substituted or unsubstituted phenyl, substituted or unsubstituted benzyl, substituted or unsubstituted vinyl, —NHR′ or —NR′R″, wherein R′ and R″ are independently H, an unsubstituted C₁ to C₆ alkyl or a C₁-C₃ halogen-substituted C₁-C₆ alkyl.

3-10. (canceled)

11. The method of claim 1, wherein the N-annulated PDI compound is: 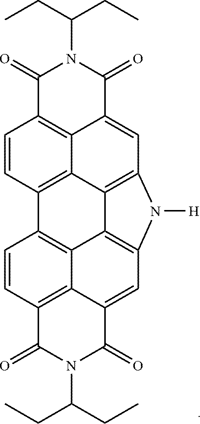
.

12. The method of claim 1, wherein the film is solvent-resistant.

13. The method of claim 1, wherein the film is resistant to water, a C₁-C₈ alcohol, a chlorinated alkane, a hydrocarbon, an aromatic hydrocarbon or an amide.

14. (canceled)

15. The method of claim 1, wherein the base is an alkyl amine, an alkali metal hydroxide, an alkaline earth metal hydroxide, an ammonium hydroxide, an alkali metal carbonate or an alkaline earth carbonate.

16-20. (canceled)

21. The method of claim 1, wherein at least one equivalent of base with respect to the pyrrole N—H bond is added.

22. The method of claim 1, wherein the solvent is a C₂-C₆ alcohol.

23-24. (canceled)

25. The method of claim 1, wherein the concentration of N-annulated PDI compound in the solvent ranges from 0.1 to 100 mg/mL.

26-27. (canceled)

28. The method of claim 1, wherein the film is formed by spin-coating, slot-die-coating or printing.

29-32. (canceled)

33. A solvent-resistant organic semiconducting film comprising non-polymeric perylene diimide molecules with a pyrrolic N—H bond or a salt thereof.

34. The solvent-resistant film of claim 33, wherein the non-polymeric perylene diimide molecule is a compound of Formula I: 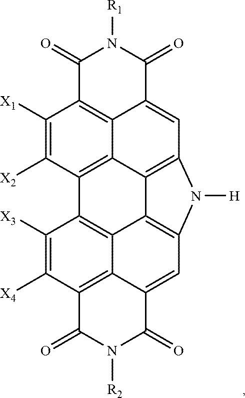
or a salt thereof, wherein:
R₁ and R₂ are independently a substituted or unsubstituted C₁ to C₁₈ linear or branched alkyl; and
X₁-X₄ are independently selected from H, a C₁-C₆ substituted or unsubstituted alkyl, a halogen, NO₂, or CN or X₂ and X₃ together form —S—S— and X₁ and X₄ are independently selected from H, a C₁-C₆ substituted or unsubstituted alkyl, a halogen, NO₂, or CN;
wherein optional substitution of alkyl groups is substitution with one or more halogens, —CN, —NO₂, —C(O)R′, —COOR′, —C(O)NH₂, —NHC(O)R′, —C(O)NR′R″, —CF₃, —SO₃H, —SO₂CF3, —SO₂R′, —SO₂NR′R′’, —OR′, —OC(O)R′, substituted or unsubstituted phenyl, substituted or unsubstituted benzyl, substituted or unsubstituted vinyl, —NHR′ or —NR′R″, wherein R′ and R″ are independently H, an unsubstituted C₁ to C₆ alkyl or a C₁-C₃ halogen-substituted C₁-C₆ alkyl.

35-37. (canceled)

38. The film of claim 34, wherein:
(1) all of X₁-X₄ are hydrogen;
(2) one of X₁-X₄ is a halogen;
(3) one of X₁-X₄ is F, Cl or Br;
(4) X₁, X₃ and X₄ are hydrogen and X₂ is a halogen, CN or an unsubstituted C₁-C₃ alkyl; or
(5) X₁, X₃ and X₄ are hydrogen and X₂ is F, Br or Cl.

39-42. (canceled)

43. The film of claim 33, wherein the perylene diimide molecule is: 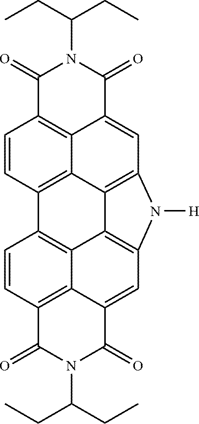 [SMILES: CCC(CC)n1c(=O)c2ccc3c4ccc5c(=O)n(C(CC)CC)c(=O)c6cc7[nH]c8cc(c1=O)c2c3c8c7c4c56]
, or a salt thereof.

44. The film of claim 33 which ranges in thickness from 10 to 1000 nm.

45-46. (canceled)

47. A film-precursor formulation which comprises a pyrrole-deprotonated N-annulated PDI compound and at least one equivalent of base to deprotonate the N-annulated PDI compound dissolved in a solvent selected from water, a C₁-C₆ alcohol or a miscible mixture thereof.

48-52. (canceled)

53. A multi-layer electronic device comprising a layer which comprises a film of claim 33.

54. The multi-layer device of claim 53 which is an organic photovoltaic (OPV) device.

55-57. (canceled)

58. A method for detecting an amine which comprises contacting a film prepared by a method of claim 1 with a gas, liquid phase or solution which may contain an amine and detecting a color change in the film indicative of the presence of an amine in contact with the film.

59. A method for making a multi-layer device of claim 53 which comprises:
forming at least one device layer on a substrate by:
dissolving a selected amount of an N-annulated perylene diimide (PDI) compound having a pyrrole N—H bond in a selected amount of a solvent selected from water, a C₁-C₆ alcohol or a miscible combination thereof by addition, to the solvent containing the selected amount of the N-annulated PDI compound, of an amount of base at least sufficient to polarize the pyrrole N—H bond giving an ionic salt of the N-annulated PDI compound dissolved in the solvent to form a film-precursor solution;
spreading, coating or printing the film-precursor solution on the substrate; and
removing solvent from film-precursor solution to form the device layer.
